(19) United States
(12) Patent Application Publication
(10) Pub. No.: US 2010/0305402 A1
(43) Pub. Date: Dec. 2, 2010

(54) METHOD AND APPARATUS FOR MAGNETIC WAVEGUIDE FORMING A SHAPED FIELD EMPLOYING A MAGNETIC APERTURE FOR GUIDING AND CONTROLLING A MEDICAL DEVICE

(75) Inventors: Yehoshua Shachar, Santa Monica, CA (US); Laszlo Farkas, Ojai, CA (US); Leslie Farkas, Ojai, CA (US)

Correspondence Address: KNOBBE MARTENS OLSON & BEAR LLP, 2040 MAIN STREET, FOURTEENTH FLOOR, IRVINE, CA, 92614, US

(73) Assignee: Magnetecs,Inc., Inglewood, CA (US)

(21) Appl. No.: 12/475,370

(22) Filed: May 29, 2009

(51) Int. Cl. A61B 1/00; H01F 3/00

(52) U.S. Cl. 600/118; 335/297

(57) ABSTRACT

A system that uses a magnetic aperture and electromagnets to configure a magnetic shaped field is described. In one embodiment, the system can be used for guiding a catheter or other devices through a patient's body. In further modification of the system, the waveguide field and field gradient is achieved by the use of varying the electromagnetic wave and its respective flux density axis. In one embodiment, one or more magnetic pole pieces (electromagnet cores) are configured with anisotropic permeability to control the shape of the resulting magnetic field. In one embodiment, the shape and permeability distribution in an electromagnet poleface is configured to produce the desired field distribution. In one embodiment, a number of electromagnets are arranged in a spherical pattern to produce a desired magnetic field in an enclosed spherical region. In one embodiment, a distal end of a catheter is provided with a plurality of magnets having different coercivity to allow improved control of the position and orientation of the distal end

### FIELD OF THE DISCLOSURE

The present invention relates generally to methods for modeling the properties of waveguides, and more particularly, to methods for generating shaped field whereby use of magnetic aperture with a specific geometry and material permeability is used.

### BACKGROUND

Catheterization is typically performed by inserting an invasive device into an incision or a body orifice. These procedures rely on manually advancing the distal end of the invasive device by pushing, rotating, or otherwise manipulating the proximal end that remains outside of the body. Real-time X-ray imaging is a common method for determining the position of the distal end of the invasive device during the procedure. The manipulation continues until the distal end reaches the destination area where the diagnostic or therapeutic procedure is to be performed. This technique requires great skills on the part of the surgeon/operator. Such skill can only be achieved after a protracted training period and extended practice. A relatively high degree of manual dexterity is also required.

The prior art extensive efforts to overcome the limitation of manually advancing the distal end of an invasive device, resulted in the establishment of a robotically guided surgical tool(s) while using magnetic force to manipulate such tool(s) for diagnostic, as well as therapeutic procedure.

Recently, magnetic systems have been proposed, wherein magnetic fields produced by one or more electromagnets are used to guide and advance a magnetically-tipped catheter. The electromagnets in such systems produce large magnetic fields that are potentially dangerous to medical personnel and can be disruptive to other equipment.

### SUMMARY

These and other problems are solved by a magnetic waveguide for guidance control of a system that uses a magnetic aperture and electromagnets to configure a magnetic shaped field for guiding a catheter or other devices through a patient's body. In further modification of the system, the waveguide field and field gradient is achieved by the use of varying the EM wave and its respective Flux density axis.

In one embodiment, a magnetic circuit is configured to generate a desired magnetic field in the region of a multi-coil cluster of electromagnets. In one embodiment, one or more poles of the cluster are modified so as to provide an anisotropic radiation with respect to other poles in the cluster, and to allow shaping of the magnetic field.

In one embodiment, one or more magnet poles are modified and the poleface geometry altered, so as to shape the magnetic field. A detailed approach to setting a mechanical analog mechanism for varying the magnetic field geometry is described by U.S. application Ser. No. 11/140,475 and is noted above. The observation and findings of testing the mechanically deployable pole-faces, in order to modify the generated field geometry, is augmented by the current application with the use of a magnetic aperture. In one embodiment, a magnetic waveguide with spherical geometry is provided with eight EM generators. The eight EM generators are further modified by the addition of an improved magnetic aperture on the pole-face of each of the EM units.

In one embodiment, the waveguide with its cluster of electromagnets can be positioned to generate magnetic fields that exert a desired torque on the catheter, but without advancing force on the tip (e.g., distal end of the catheter). This affords bend and rotate movements of the catheter tip toward a selected direction.

In one embodiment, the multi-coil cluster is configured to generate a relatively high gradient field region for exerting a moving force on the tip (e.g., a push-pull movement), with little or no torque on the tip.

In one embodiment, the waveguide forming the magnetic chamber includes a closed-loop servo feedback system.

Another embodiment of the waveguide magnetic chamber is configured as a magnetic field source (the generator) to create a magnetic field of sufficient strength and orientation to move a magnetically-responsive surgical tool(s) such as catheter-tip to provide manipulation of the tool in a desired direction by a desired amount.

In one embodiment, a Detection System 350, as noted in Shachar U.S. Pat. No. 7,280,863, is described by the use Radar and other imaging modalities so as to identify the location and orientation of surgical tool(s) within a patient's body. The Radar employs the principle of dielectric properties discrimination between biological tissue-dielectric constant vs. the dielectric properties of polymers, metals or other synthetic materials forming the medical tool, while further establishing the Spatial as well as Time domain differentiating signal due to conductivity and attenuation in mixed media. Position detection using Impedance technique, Hall Effect Sensor, or other means of magnetic positioning techniques are detailed by Shachar et al. patents applications noted above for reference.

In one or more embodiments, the mode used for determining the location of the distal end of the surgical tool(s) or catheter like device inside the body minimizes or eliminates the use of ionizing radiation such as X-rays, by allowing magnetic waveguide apparatus to scale the magnetic force or force gradient to the appropriate amount relative to tool position and orientation.

In one embodiment, the use of scalability rules are identified, and a scale model 1, was built in order to demonstrate the performance of waveguide's ferro-refraction magnification technique and the use of hybrid permeability poleface. Scale model is used so as to experimentally demonstrate the embodiments.

The scale model reference designator 1 is a 2D four coil assembly which is expended to a 3D geometry by the use of topological transformations. The transformations from a four coil circuit symmetry to an eight coil spherical symmetry is noted by FIGS. 13A through 13D with its accompanying description. The resultant spherical topology provides for the construction of a waveguide while preserving linearity under vector field operation.

Further embodiments of the scale rules guiding the construction of scale model 1 are the tailoring of constants relating to geometrical orientation of the polefaces so as to modify the anisotropic radiation of the EM generators and provide for optimization of flux density axis location relative to the location of the tool magnetic tip.

Scaling rules regulate the appropriate magnetic forces exerted by the waveguide relative to the actual (AP) vs. desired position (DP). The drawings and accompanying specifications will instruct the reader on the use and application of these rules when applied to the art of regulating magnetic force, and by forming such field under guidelines governing optical effects, such as noted in this application; ferro-refraction, total internal reflection, the formation of magnetic aperture with hybrid permeability values, and others principles articulated by this application.

In one embodiment, the waveguide multi-coil cluster is configured to generate a magnetic field gradient for exerting an orthogonal force on the tip (side-ways movement), with little or no rotating torque on the tip. This is useful, for example, to align the catheter's tip at narrow forks of artery passages and for scraping a particular side of artery or in treatment of mitral valve stenosis.

In one embodiment, the waveguide multi-coil cluster is configured to generate a mixed magnetic field to push/pull and/or bend/rotate the distal end of the catheter tip, so as to guide the tip while it is moving in a curved space and in cases where for example the stenosis is severe or artery is totally blocked.

In one embodiment, the waveguide multi-coil cluster is configured to move the location of the magnetic field in 3D space relative to a desired area. This magnetic shape control function provides efficient field shaping to produce desired magnetic fields, as needed, for example, in surgical tool manipulations in the operating region (herein defined as the Effective Space).

One embodiment employs the waveguide with its Shaped Magnetic Regulator to position the tool (catheter tip) inside a patient's body, further maintaining the catheter tip in the correct position. One embodiment includes the ability of the waveguide regulator to steer the distal end of the catheter through arteries and forcefully advance it through plaque or other obstructions.

In one embodiment, the physical catheter tip (the distal end of the catheter) includes a permanent magnet that responds to the magnetic field generated externally by the waveguide. The external magnetic field pulls, pushes, turns, and holds the tip in the desired position. One of ordinary skill in the art will recognize that the permanent magnet can be replaced or augmented by an electromagnet.

One embodiment includes the waveguide and its regulating apparatus that is more intuitive and simpler to use, that displays the catheter tip location in three dimensions, that applies force at the catheter tip to pull, push, turn, or hold the tip as desired, and that is configured to producing a vibratory or pulsating motion of the tip with adjustable frequency and amplitude to aid in advancing the tip through plaque or other obstructions. One embodiment provides tactile feedback at the operator control to indicate an obstruction encountered by the tip. In one embodiment, the amount of tactile feedback is determined based, at least in part, on a difference between the actual position and the desired position. In one embodiment, the amount of tactile feedback is determined based, at least in part, on the strength of the applied magnetic field used to move the catheter tip. In one embodiment, tactile feedback is provided only when the position error (or applied field) exceeds a threshold amount. In one embodiment, tactile feedback is provided only when the position error exceeds a threshold amount for a specified period of time. In one embodiment, the amount of tactile feedback is determined based at least in part on a difference between the actual position and the desired position.

One embodiment of the waveguide and its regulator includes a user input device called a “virtual tip” (VT). The virtual tip includes a physical assembly, similar to a joystick, which is manipulated by the surgeon/operator and delivers tactile feedback to the surgeon in the appropriate axis or axes if the actual tip encounters an obstacle. The Virtual Tip includes a joystick type device that allows the surgeon to guide actual surgical tool such as catheter tip through the patient's body. When actual catheter tip encounters an obstacle, the virtual tip provides tactile force feedback to the surgeon to indicate the presence of the obstacle.

In one embodiment, the waveguide symmetry (e.g., eight coil cluster) configuration, which allows a regulator to compute the desired field(s) under the doctrine of linear transformation of matrices in the magnetic chamber so as to provide closure of all vector field operations (addition, subtraction, superposition, etc.) without the need for tailoring the waveguide-regulator linearity. This symmetry provides within the effective space.

In one embodiment, the physical catheter tip (the distal end of the catheter) includes a permanent magnet and/or multiple articulated permanent magnets so as to provide manipulation of the distal end of a surgical tool by the use of the waveguide to generate mixed magnetic fields. The use of multiple permanent magnetic elements with different coercivity (H_{cJ}) values, will result in a “primary bending mode” and a “secondary bending mode” on the same axis (relative to the EM field axis), while using, for example, on the one hand a Sintered Nd—Fe—B {near net-shape magnets with a high remnant polarization of 1.37 T, and a coercivity H_{cJ} of 9.6 kA/cm (12 kOe), and a maximum energy density of 420 kJ/m³ (53 MGOe)},and on the other hand a secondary permanent magnet(s) adjacent to the distal one with a coercivity H_{cJ} of 6.5 kA/cm.

The embodiment of Mixed Magnetic Field provides the waveguide with the ability to employ the inherent anisotropic behavior of the EM field as well as the EM wave influence on the inherent properties of the surgical tool(s), within the waveguide chamber, resulting in formation of universal magnetic joint facilitating guidance and control of the catheter in complex geometry.

In one embodiment, the waveguide EM circuit includes a C-arm geometry using a ferromagnetic substance, such as parabolic antenna, (e.g., a magnetic material, such as, for example, a ferrous substance or compound, nickel substance or compound, cobalt substance or compound, etc.) further increasing the efficiency of the waveguide as the electro-magnetic field's energy is attenuated by the parabolic shielding antenna which forms an integral flux carrier and provides containment of stray fields.

In one embodiment, the waveguide regulator uses numerical transformations to compute the currents to be provided to various electromagnets so as to direct the field by further positioning one or more of the electromagnet to control the magnetic field used to push/pull and rotate the catheter tip in an efficient manner within the chamber.

In one embodiment, the waveguide regulator includes a mechanism to allow the electromagnet poles faces to form a shaped magnetic based on a position and orientation of the catheter's travel between the DP and AP. This method is further optimizing the necessary power requirements needed to push, pull, and rotate the surgical tool tip. By employing “lensing” modes of the field with the use of a magnetic Aperture, the waveguide forms a shaped magnetic field relative to the minimal path between AP to DP.

In one embodiment, the waveguide is fitted with sensory apparatus for real time (or near real time) detection of position and orientation so as to provide command inputs to a servo system that controls the tool-tip location from AP to DP. The desired position, further generates a command which results in shaping the magnetic field geometry based on magneto-optical principles as shall be clear when reviewing the figures and the accompanying descriptions.

In one embodiment, the waveguide's servo system has a correction input that compensates for the dynamic position of a body part, or organ, such as the heart, thereby offsetting the response such that the actual tip moves substantially in unison with the dynamic position (e.g., with the beating heart). Further, synchronization of dynamic position of a surgical tool with the appropriate magnetic field force and direction is accomplished by the response of the waveguide regulator and its resulting field's intensity and field's geometry.

In one embodiment, the waveguide magnetic chamber, its regulator and a magnetically fitted tool, are used in a system where: i) the operator adjusts the physical position of the virtual tip (VT), ii) a change in the virtual tip position is encoded and provided along with data from a position detection system, iii) the regulator generates servo system commands that are sent to a servo system control circuitry, iv) the servo system control apparatus operates the servo mechanisms to adjust the condition of one or more electromagnet from the cluster by varying the power relative to distance and/or angle of the electromagnet clusters vis-a-vie the tool's permanent magnet position, further energizing the electromagnets so as to control the magnetic (catheter) tip within the patient's body, v) the new position of actual catheter tip is then sensed by the position detection system, thereby allowing for example a synchronization of the catheter position on an image produced by fluoroscopy (and/or other imaging modality, such as, for example, ICE, MRI, CAT or PET scan), vi) and the like to provide feedback to the servo system control apparatus and to the operator interface and vii) updating the displayed image of the catheter tip position in relation to the patient's internal body structures.

In one embodiment, the operator can make further adjustments to the virtual catheter tip (VT) position and the sequence of acts ii through vii above is repeated. In one embodiment, the feedback from the servo system and control apparatus (the regulator), deploys command logic (AI routine) when the actual catheter tip encounters an obstacle or resistance in its path. The command logic is further used to control stepper motors which are physically coupled to the virtual catheter tip. The stepper motors are engaged so as to create resistance in appropriate directions that can be felt by the operator, and tactile feedback is thus provided to the user.

In one embodiment, the regulator uses scaling factors to calculate the magnetic field generated along the waveguide effective magnetic space.

In one embodiment, the waveguide generates a maximum torque of 0.013 Newton-meter on the tool's tip, while the coil cluster is generating a magnetic field strength between B=0.04T and 0.15T.

In one embodiment, the coil current polarity and polarity rotation are configured to allow the coil cluster to generate torque on the catheter tip.

In one embodiment, the coil current polarity and rotation are configured to provide an axial and/or orthogonal force on the catheter.

In one embodiment, the waveguide eight-coil symmetry provides for an apparatus that generates the desired magnetic field in an optimized pattern.

In one embodiment, the waveguide with its coil cluster is fitted with a parabolic shield (the magnetic shield antenna), collecting the magnetic flux from the effective space and creates a return path to decrease the need to shield the stray magnetic radiation beyond the waveguide 3D metric footprint.

In one embodiment, the waveguide magnetic circuit efficacy is evaluated as to its topological properties (symmetry, linearity) and is measured relative to torque control and field variations of flux densities within the effective space.

In one embodiment, the waveguide magnetic circuit efficacy is evaluated as to its topological properties and is measured relative to force control gradient variations in the ±80 mm region around the magnetic center (field stability and uniformity).

In one embodiment, the waveguide-regulator with its rotational transformation and its relationship to field strength and field gradient are mathematically established. This embodiment forms the core competency of the regulator to establish a predictable algorithm for computing the specific field geometry with the associated flux density so as to move the catheter tip from AP to DP.

In one embodiment, a ferro-refraction technique for field magnification is obtained when a current segment is near a high magnetic permeable boundary. The ferro-refraction can enhance the design and performance of magnets used for NMR or MRI by increasing the efficiency of these magnets. Ferro-refraction refers to the field magnification that can be obtained when a current segment is near a high magnetic permeability (μ) boundary. Refraction occurs at any boundary surface between two materials of different permeability. At the surface, the normal components of the magnetic induction (B) are equal, while the tangential components of the magnetic field (H) are equal.

In one embodiment, waveguide magnification of the field is improved by the magnetic aperture poleface material permeability and its anisotropic behavior to form a suitable lens for establishing an efficient geometry and flux density for guiding and controlling the movement of the catheter tip from AP to DP. This enhancement is guided analytically by the Biot-Savart law and the inclusion of mirror image currents. (See: An Open Magnet Utilizing Ferro-Refraction Current Magnification, by, Yuly Pulyer and Mirko I. Hrovat, Journal of Magnetic Resonance 154, 298-302 (2002).

In one embodiment, a mathematical model for predicting the magnetic field geometry (Shaped) versus magnetic field strength is established relative to the catheter tip axis of magnetization and is used by the waveguide regulator to predict and command the movements of a surgical tool from its actual position (AP) to its desired position (DP).

In the particular applications of using a magnetically guided catheter the waveguide principle is used for forming a bounded, significant size electromagnetic chamber, within which controllable energy propagation can take place. In contrast to HF waveguides, the chamber of a spherically confined magnetic field generator requires not only directional field-power flow, but this flow needs to be three-dimensional. Energy in the generated field is then transferred through the electromagnetic interaction between the field and the guided catheter, providing the work to move and propel a medical tool(s) such as catheter from Actual Position (AP) to Desired Position (DP) while negotiating such translational, as well as, rotational forces against blood-flow, tissue forces and catheter stiffness is optimized.

The magnetic field generator, having multiple core-coils located around the operating area (effective space), shapes the chamber magnetic field to establish a three dimensional energy propagation wavefront which can be stationary as well as can be moved and shaped to provide the necessary power flow into the distal end of magnetic catheter tip so as to torque it and/or push it in the direction of the power flow. In a closed location and direction with control loop, such that the desired position (DP) of the catheter tip can be then obtained.

The field generator has two or more modes of operation. In one mode, it generates a static magnetic field which stores the guidance energy in the operating region in accordance with the following equation:

$${U}_{static} = {\int }_{0}^{B} H \cdot d B [ J / {cm}^{3} ] \qquad 1 )$$

This energy produces the work of transporting the tip magnet 7, from AP location to the DP. This work relates to the magnetic field as follows:

$$W = - k {\int }_{0}^{b} H \cdot d l \qquad 2 )$$

Where k is the factor which combines magnetic and physical constants.

The static fields are generated as the result of the superposition of multiple static magnetic fields and are shaped and focused to produce the required field strength and gradient to hold the catheter tip in a static position and direction. The system satisfies the Maxwell's equations for static magnetic field.

Once the catheter tip needs to move or change direction, the system operates in the dynamic mode which involves time varying transient field conditions. In this mode, the time varying form of the Maxwell's equations need to be used in assessing the waveguide capabilities for controlling the electromagnetic transient propagation of the EM (electromagnetic) energy in the chamber while using the multi-coil magnetic radiator assembly (the waveguide).

These transient dynamic conditions are described by the Wave Equations:

$$\begin{matrix}{∇}^{2} E = {k}_{2} ( \frac{σ}{ɛ} \frac{δ H}{δ t} + \frac{{\partial }^{2} H}{\partial {t}^{2}} ) \\ ∇ E = 0 & 3 ) \\ {∇}^{2} M = {k}_{3} ( \frac{σ}{ɛ} \frac{δ H}{δ t} + \frac{{\partial }^{2} H}{\partial {t}^{2}} ) \\ ∇ M = 0 & 4 )\end{matrix}$$

In one embodiment, the field distributions satisfy these field equations in addition to Maxwell's formalism. During the dynamic regulations the linear superimposition entails the calculations of longitudinal propagation of waves generated from each source. The longitudinal components are extracted from the wave equation by solving the following differential equation:

$$\frac{{\partial }^{2} {H}_{z}}{\partial {x}^{2}} + \frac{{\partial }^{2} {H}_{Z}}{\partial {y}^{2}} = - {k}_{4}^{2} {H}_{z} \qquad 5 )$$

The energy in the dynamic field can then be calculated:

$${U}_{dynamic} = \frac{ɛ}{2} \int {E}^{2} d τ + \frac{μ}{2} \int {H}^{2} d τ \qquad 6 )$$

And the power in the propagated wave:

P_{wave}=∫(E×H)·dS 7)

The electric E component at the field regulation speeds required for catheter guidance is relatively small in comparison to the magnetic component. However, the superposition of the complementary electromagnetic fields generated by a pair of spherically symmetric core-coil pairs will generate a field which behaves as a standing wave, dynamically changing the three-dimensional magnetic field at and around the center of the operating region (effective space 10).

A scale model 1 is used herein to explain magnetic field shaping and description of the diagnostic and therapeutic procedure while employing a catheter within a patient's body organ.

The waveguide as a magnetic field generator, with approximately 80 mm diameter, with spherical chamber within the operating region-(the effective space) is described. The objective of the waveguide structure is to generate about 0.10 Tesla field strength and about 1.3 Tesla/meter field gradient in this region exerting adequate torque and force on a 2.30 mm diameter×12 mm long (7 Fr) permanent magnet installed at the tip of a surgical catheter. Magnetic focusing reduces the field generator size, weight and power consumption.

Techniques disclosed herein to concentrate the field in the center operating region include:
a) Shaped and oriented magnetic polefaces,-magnetic aperture geometry, hereinafter defined by reference designator, 4.x.
b) Anisotropic permeability built into the polefaces, Magnetic aperture material, hereinafter defined by reference designator, 5.x.
c) Magnetic containment using shield-like magnetic returns integrated into the outer surface of the magnetic field generator,-waveguide parabolic antenna, hereinafter defined by reference designator, 18.

The first two techniques combined exhibit and defined the flux refractory behavior along the rules governing an optical lens behavior, while observing visible light transmission through different refractory index. Hence, the use of an apparatus and method in forming a magnetic aperture within the confinement of a waveguide is described to provide magnetic lensing.

In another embodiment, the permeability of the magnetic material can be varied electronically, thus a dynamic aperture correction can be devised producing the needed field parameters in the operating region with reduced field generator power.

In another embodiment, the optical behavior of ferrous materials having negative permeability at or near permeability resonance can yield large field amplifications and can refract flux lines through negative angles.

One embodiment includes an apparatus for controlling the movement of a catheter-type tool inside a body of a patient, including a magnetic field source for generating a magnetic field, the magnetic field source including a first coil disposed to produce a first magnetic field in a first magnetic pole piece and a second coil disposed to produce a second magnetic field in a second magnetic pole piece, the first magnetic pole piece including a first anisotropic permeability that shapes the first magnetic field; the second magnetic pole piece including a second anisotropic permeability that shapes the second magnetic field, the first magnetic pole piece and the second magnetic pole piece disposed to produce a shaped magnetic field in a region between the first magnetic pole piece and the second magnetic pole piece; and a system controller for controlling the magnetic field source to control a movement of a distal end of a catheter, the distal end responsive to the magnetic field, the controller configured to control a current in the first coil, a current in the second coil, and a position of the first pole with respect to the second pole.

One embodiment includes the system controller including a closed-loop feedback servo system.

One embodiment includes the first magnetic pole piece including a body member and a field shaping member, the field shaping member disposed proximate to a face of the first pole piece, the body member including a first magnetic material, the field shaping member including a second magnetic material different from the first magnetic material.

One embodiment includes a first magnetic pole piece including a body member and a field shaping member, the field shaping member disposed proximate to a face of the first pole piece, the body member including a first magnetic material composition, the field shaping member including a second magnetic material different from the first magnetic material composition.

In one embodiment, the second magnetic material composition includes an anisotropic permeability.

In one embodiment, the first magnetic pole piece includes a face including a concave depression.

In one embodiment, the first magnetic pole piece includes a face having a first concave depression and the second magnetic pole piece includes a face having a second concave depression, the shaped field formed in a region between the first concave depression and the second concave depression.

In one embodiment, the first magnetic pole piece includes a core member including a first magnetic material composition and a poleface member disposed about the magnetic core including a second magnetic material composition.

In one embodiment, the poleface member is substantially cylindrical.

In one embodiment, the first magnetic pole piece includes a substantially cylindrical core including a first magnetic material composition and a poleface cylinder disposed about the magnetic core including a second magnetic material composition.

In one embodiment, the substantially cylindrical core extends substantially a length of the first magnetic pole piece.

In one embodiment, a cylindrical axis of the first magnetic pole piece is disposed substantially parallel to a cylindrical axis of the second magnetic pole piece.

In one embodiment, the distal end includes a permanent magnet.

In one embodiment, the distal end includes an electromagnet.

In one embodiment, the distal end includes a first magnet having a first coercivity and a second magnet having a second coercivity.

In one embodiment, the first magnetic pole piece includes a first magnetic material and wherein the system controller includes a control module to control a permeAbility of the first magnetic material.

In one embodiment, the servo system includes a correction factor that compensates for a dynamic position of an organ, thereby offsetting a response of the distal end to the magnetic field such that the distal end moves in substantial unison with the organ.

In one embodiment, the correction factor is generated from an auxiliary device that provides correction data concerning the dynamic position of the organ, and wherein when the correction data are combined with measurement data derived from the sensory.

In one embodiment, the auxiliary device is at least one of an X-ray device, an ultrasound device, and a radar device.

In one embodiment, the system controller includes a Virtual Tip control device to allow user control inputs.

One embodiment includes a first controller to control the first coil; and a second controller to control the second coil. In one embodiment, the first controller receives feedback from a magnetic field sensor.

In one embodiment, the system controller coordinates flow of current through the first and second coils according to inputs from a Virtual Tip. In one embodiment, the Virtual Tip provides tactile feedback to an operator when a position error exceeds a threshold value. In one embodiment, the Virtual Tip provides tactile feedback to an operator according to a position error between an actual position of the distal end and a desired position of the distal end. In one embodiment, the system controller causes the distal end to follow movements of the Virtual Tip.

One embodiment includes a mode switch to allow a user to select a force mode and a torque mode.

One embodiment includes an apparatus for controlling the movement of a catheter-like tool to be inserted into the body of a patient, including: a controllable magnetic field source having a first cluster of poles and a second cluster of poles, wherein at least one pole in the first cluster of poles includes an anisotropic pole piece, the anisotropic pole piece including a core member and a poleface member, the core member and the poleface member including different compositions of magnetic material, the first cluster of poles and the second cluster of poles disposed to direct a shaped magnetic field in a region between the first cluster of poles and the second cluster of poles; a first group of electromagnet coils provided to the first cluster of poles and a second group of electromagnet coils provided to the second cluster of poles; and a controller to control electric currents in the first group of electromagnet coils and the second group of electromagnet coils to produce the shaped magnetic field.

In one embodiment, the poleface member includes a substantially concave face.

In one embodiment, the controller controls a permeability of the poleface member.

In one embodiment, the first cluster of poles is coupled to the second cluster of poles by a magnetic material.

One embodiment includes calculating a desired direction of movement for the distal end, computing a magnetic field needed to produce the movement, the magnetic field computed according to a first bending mode of the distal end and a second bending mode of the distal end, controlling a plurality of electric currents and pole positions to produce the magnetic field, and measuring a location of the distal end.

One embodiment includes controlling one or more electromagnets to produce the magnetic field.

One embodiment includes simulating a magnetic field before creating the magnetic field.

One embodiment includes controlling the movement of a catheter-like tool having a distal end responsive to a magnetic field and configured to be inserted into the body of the patient, including a magnetic field source for generating a magnetic field, the magnetic source including an electromagnet, the electromagnet including an electromagnet coil, a pole piece core, and a poleface insert, the poleface insert having a different permeability than the pole piece core, a sensor system to measure a location of the distal end, a sensor system to measure positions of a plurality of fiduciary markers, a user input device for inputting commands to move the distal end, and a system controller for controlling the magnetic field source in response to inputs from the user input device, the radar system, and the magnetic sensors. One embodiment includes a closed-loop feedback servo system.

In one embodiment, the poleface insert is disposed proximate to a face of the pole piece core.

In one embodiment, the distal end including one or more magnets.

In one embodiment, the distal end including a first magnet having a first coercivity and a second magnet having a second coercivity.

In one embodiment, the system controller calculates a position error and controls the magnetic field source to move the distal end in a direction to reduce the position error.

In one embodiment, the system controller computes a position of the distal end with respect to a set of fiduciary markers.

In one embodiment, the system controller synchronizes a location of the distal end with a fluoroscopic image.

In one embodiment, a correction input is generated by an auxiliary device that provides correction data concerning a dynamic position of an organ, and wherein the correction data are combined with measurement data from the radar system to offset a response of the control system so that the distal end moves substantially in unison with the organ.

In one embodiment, the auxiliary device includes, at least one of, an X-ray device, an ultrasound device, and a radar device.

In one embodiment, the user input device includes a virtual tip control device to allow user control inputs.

In one embodiment, a virtual tip provides force feedback.

In one embodiment, a first coil cluster is fitted with shield for flux return.

### BRIEF DESCRIPTION OF THE DRAWINGS

FIG. 1 is an orthographic cross-section of the apparatus forming the magnetic aperture and its EM radiator.

FIG. 1A is an orthographic representation of a magnetic aperture and the resultant flux line geometry.

FIG. 1B is an orthographic representation of the refraction index generated by a magnetic aperture.

FIG. 1C is a graphic depiction the magnetic aperture geometry layout.

FIG. 1D is a graphic representation of the EM generator (electromagnet assembly).

FIG. 2 is an orthographic depiction of the directional and flux density map.

FIG. 2A is an orthographic depiction of the Poleface cylindrical insert layout.

FIG. 2B is a graphic representation of the directional and flux density map with relative permeability constants.

FIG. 2C is a graphic representation of the magnetic aperture with a hybrid permeability aperture.

FIG. 2D is a view depicting the magnetic aperture with a hybrid permeability values.

FIGS. 3, 3A, 3B and 3C are graphic representations of the waveguide scale model.

FIGS. 4, 4A, and 4B are representations of the magnetic rules governing the waveguide performance.

FIGS. 4C and 4D are icons describing the torque and force magnetic matrices.

FIGS. 5, 5A, and 5B illustrate the vector field plot of the B fields in a central region of the waveguide.

FIGS. 6, 6A, and 6B further illustrate a case where the B vector is parallel to the −Y axis.

FIGS. 7, 7A, and 7B illustrate the waveguide and the matrix algorithm for torque mode.

FIGS. 8, 8A, and 8B illustrate the behavior of the scale model in force control mode.

FIGS. 9, 9A, and 9B illustrate the force control mode orthogonal to the magnet axis.

FIGS. 10, 10A and 10B illustrate the scale model force control mode demonstrating the use of poleface with core extension.

FIG. 11 shows a four-coil formation with magnetic core extensions.

FIG. 11A shows the core coil 1A with its core withdrawn, forming a new geometry.

FIG. 11B shows the shaped magnetic field when the core on the coil is retracted.

FIGS. 12 and 12A show the waveguide and a configuration of the magnetic field geometry under rotation condition.

FIGS. 12B, 12C, 12D, 12E, 12F and 12G are graphic depictions of various states of the waveguide performance as a combination of direction as well as power intensities is demonstrated.

FIGS. 13A, 13B, 13C, and 13D are isometric representations of the waveguide topology transformations.

FIGS. 14A, 14B and 14C are orthographic representations of the medical tool(s) such as a catheter.

FIG. 15 is a perspective view showing one embodiment of the Virtual Tip.

FIGS. 16 and 16A illustrate the field regulator loop.

FIGS. 17A, 17B and 17C are orthographic representations of the waveguide mechanical elements and magnetic circuit forming the waveguide chamber.

FIGS. 18A and 18B are isomorphic depictions of the waveguide assembly formed out of four segments of a spherical chamber.

FIG. 19 shows the waveguide with an 8 coil cluster with parabolic antenna shield.

FIG. 19A is an illustration of the B fields generated by the 8 coil cluster with the parabolic antenna shield.

FIG. 19B is an illustration of the B fields generated by the 8 coil cluster with the parabolic antenna shield.

FIG. 20 is a block diagram describing the relation between the functional elements described herein.

### DETAILED DESCRIPTION

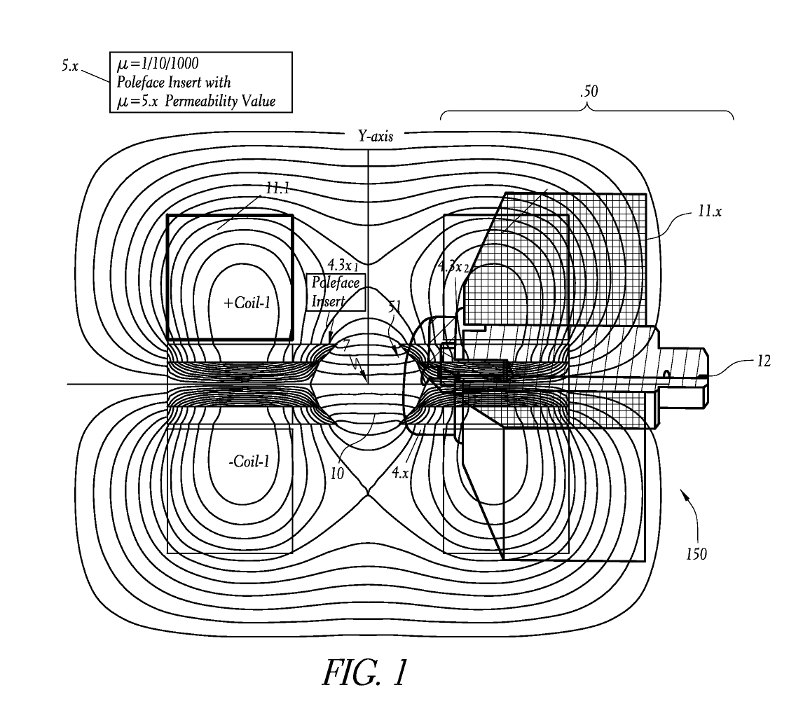

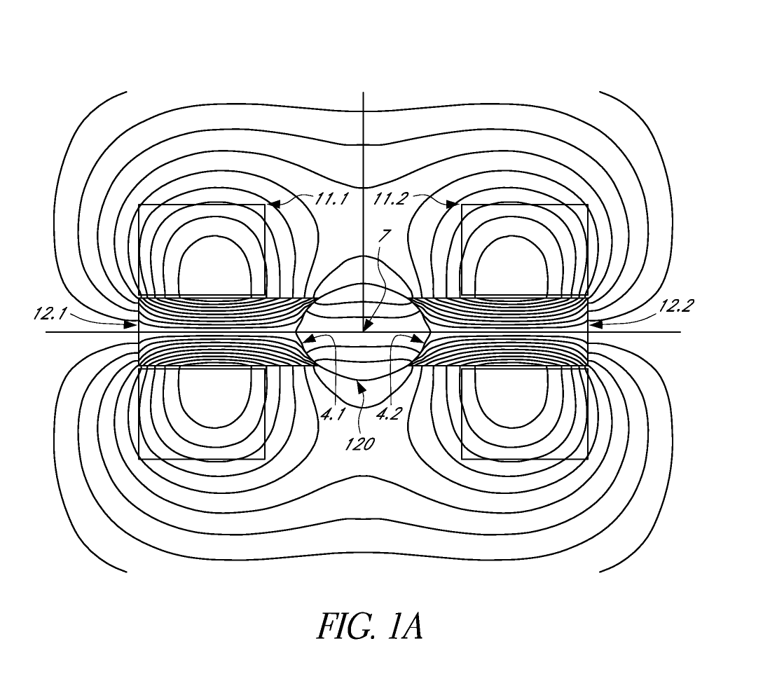

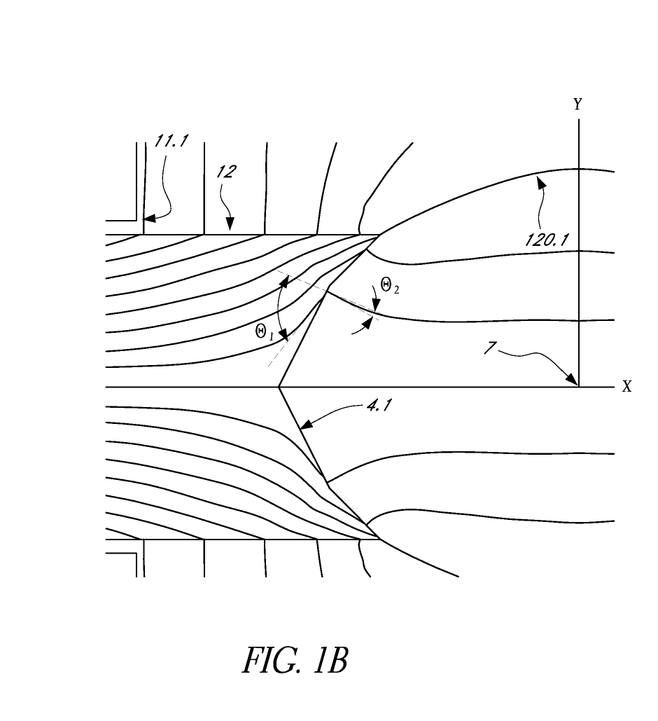

FIG. 1 is an orthographic cross section of an electromagnet 150 including a coil 11.x and a magnetic core (pole piece) 12. The magnetic core 12 has a cross section “aperture” 50 with magnetic permeability that varies across the aperture (cross section) of the core 12 to produce a desired magnetic flux configuration at a poleface 51. The end region of pole piece proximate to the effective region 10 is referred to as the poleface 51. The shape of the poleface and the construction and composition of the cores 12, 12.1, 12.2, etc., are used to a desired magnetic flux geometry in the aperture 50. As shown below, the variation of the magnetic permeability of the core 12 can be produced in various ways, such as, for example: constructing the core 12 as an inner core and one or more concentric cylinders having different permeability; constructing the core 12 using a first ferromagnetic material and providing one or more pole pieces disposed proximate to the poleface 51, combinations of these, etc. The variation of the magnetic permeability of the core 12 can also be produced by varying the material composition of various portions of the core 12 such that different portion of the aperture 50 (e.g., the core cross section) have different material composition and thus different permeability, etc.

In one embodiment, the permeability of the core 12 is controlled proximate to the face 51 such that the permeability changes radially with respect to the center of the face 51. In one embodiment, the permeability of the core 12 is controlled such that the permeability in regions closer to the axial centerline of core 12 (e.g., regions nearer to the central region of the face 51) is relatively greater than the permeability of one or more regions further from the axial centerline of the core 12 (e.g., regions nearer the outer portions of the face 51).

FIGS. 1A and 1B are schematic representations a waveguide 100 and the magnetic aperture 50. Discontinuity and/or variations of material properties, such as the permeability of the ferrous materials used in the magnetic field generator; coil 11.1, core 12.1, and poleface 4.1, and air, within the operating region changes the refractive angle at the boundaries as the flux leaves the ferrous material and enters the operating region 10. In the case of a dual core-coil arrangement (shown in FIG. 1A, ref. designators 11.1, 11.2 and 12.1, 12.2), fitted with concave polefaces 4.1 and 4.2, the flux is directed back to the operating region focusing the flux distribution while forming a lens geometry. The lens geometry ref. designator 5._{X1} is indicative of the possible insertion of multiple geometric forms in support of different field configurations, and as it is further illustrated by the flux line configuration 120.1. In one embodiment, the relative permeabilities of the ferrous materials used in the magnetic field generator are greater than 1000.

The flux lines generated (e.g.120.1) by the current in one coil 11.1 is not close around the coil directly, but are bending so as to follow the path through the core 12.1 of the other coil 11.2 and its core 12.2.

The general laws of electromagnetic wave propagation through materials of different dialectic and magnetic properties are described by Snell's law of refraction. In its simplest form, the law states that the relative angles of wave propagation in one media through the boundary of the second media depends on both the dielectric and magnetic properties of each media, jointly defining the index of refraction coefficient n(ω). The speed of the electromagnetic wave is given by c, thus the speed of magnetic wave propagation in the media is inversely proportional to the index of refraction. This index can be expressed in terms of permittivity ε(ω) and μ(ω). The permittivity and permeability of the mediums are related to the index of refraction by the relation of μ(ω)·ε(ω)=n²(ω)/c². Now the Snell's law states:

n₁ sin(θ₁)=n₂ sin(θ₂) 8)

In a static (ω≅0) magnetic structure one can write for the general relation:

$$\frac{{B}_{1 t}}{{μ}_{1}} = \frac{{B}_{2 t}}{{μ}_{2}} if {J}_{s} = 0 \qquad 9 )$$

where subscript 1t and 2t stands for the tangential components of B on both sides of the boundary. The tangential components of B are discontinuous regardless of any current density at the interface. This discontinuity is related to the permeability of the two mediums.

As a consequence of the above interface conditions, the magnetic field (either H or B) is refracted at the interface between the two materials (magnetic steel and air) with different permeability (μₛₜₑₑₗ→1000 and μₐᵢᵣ=1)

$$tan {θ}_{1} = \frac{{H}_{1 t}}{{H}_{1 n}} and tan {θ}_{2} = \frac{{H}_{2 t}}{{H}_{2 n}} \qquad 10 )$$

where t stands for tangential component and n for normal component. Substituting H=B/μ and B₁ₙ=B₂ₙ yields

$$\frac{tan {θ}_{1}}{tan {θ}_{2}} = \frac{{μ}_{1}}{{μ}_{2}} \qquad 11 )$$

Equations [8] and [11] correspond to a common interpretation of a relativistic wave propagation dynamics and its salient case of a non-relativistic static perspective. The static solution derived from FIG. 1B calculates as follows:

$${θ}_{1} = 80 ° {μ}_{1} = 1000 {μ}_{2} = 1 \\ tan {θ}_{2} = \frac{{μ}_{2}}{{μ}_{1} \cdot tan {θ}_{1}} thus {θ}_{2} < 1 ° \qquad 12 )$$

Thus, the magnetic flux exits the pole face 4.X relatively closer to perpendicular pointing from the concave-shaped surface (Magnetic Aperture 4.1 and 4.2), into the operating region 10. A further improvement, and another embodiment of the above shaped poleface focusing, is to add a cylindrical core-ring 12.1 and 12.2 to the otherwise isotropic magnetic steel core of coils 11.1 and 11.2. In one embodiment, the added core 12.x, has a relative permeability value μ=10. This embodiment of varying the permeability values, by incorporating different materials with variable g. This anisotropy in magnetic properties can be used to shape the resulting magnetic field(s) geometry as desired.

FIG. 1C shows a flux line geometry in the region 10 between a magnetic core 12.1 and a magnetic core 12.2. The construction of the cores 12.1 and 12.2 focus the magnetic field by magnetic lensing to narrow the trajectory of the field lines between the cores 12.1 and 12.2. The construction of the cores 12.1 and 12.2 and the shape of the faces of the cores 12.1 and 12.2 bends the flux lines toward the center of the region 50 allowing focused enhancement of the flux density in the central portion of region 50. The magnetic core 12.1 includes an inner core 4.1 having a first magnetic permeability and an outer cylinder 4.31 (also referred to as a poleface cylinder) having a second magnetic permeability. Similarly, the magnetic core 12.2 includes an inner core 4.2 having a first magnetic permeability and an outer cylinder (also referred to as a poleface cylinder) 4.32 having a second magnetic permeability. One of ordinary skill in the art will recognize that it is convenient for the inner cores 4.1, 4.2 and the outer cylinders 4.31, 4.32 to have generally cylindrical cross sections, but that such is not required and the inner cores 4.1, 4.2 and outer cylinders 4.31, 4.32 can be constructed with different cross sections, such as for example, oval, polygonal, etc.

Due to the anisotropy of the magnetic permeability across the core 12.1, 12.2, the flux density increases in the central region. Typically the relative permeability, of the inner core material 4.2 is greater than the relative permeability μᵣ of the material of the outer cylinder 4.32. In one embodiment, the cores 12.1 and 12.2 include an inner core 4.2 of μᵣ=1000, a outer cylinder 4.32 with μᵣ=10 to produce the desired flux field in the region of air (with μᵣ=1) between the cores 12.1 and 12.2.

FIG. 1D is an orthographic depiction of the core/coil and the magnetic aperture 50, forming the electromagnet EM generator 17.x (the x-index the relative position of the eight EM generators on the waveguide stricture). FIG. 1D view “A” shows the EM generator 17, with its magnetic aperture 4.x, its low permeability ring insert 4.3x_{y}. The generator in view “B”, indicate a cutoff illustrating the coil 11.x, the core 12.x, followed in view “C” by indicating the isometric view of the insert ring 4.3x_{y}. The figure further shows a schematic of the generator 17.

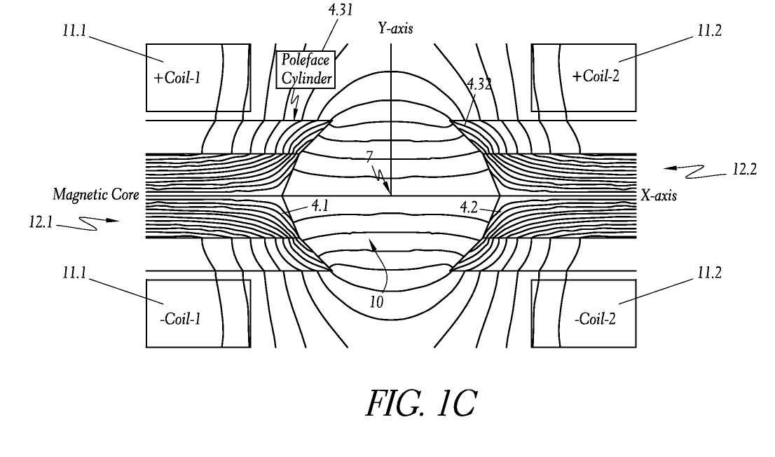

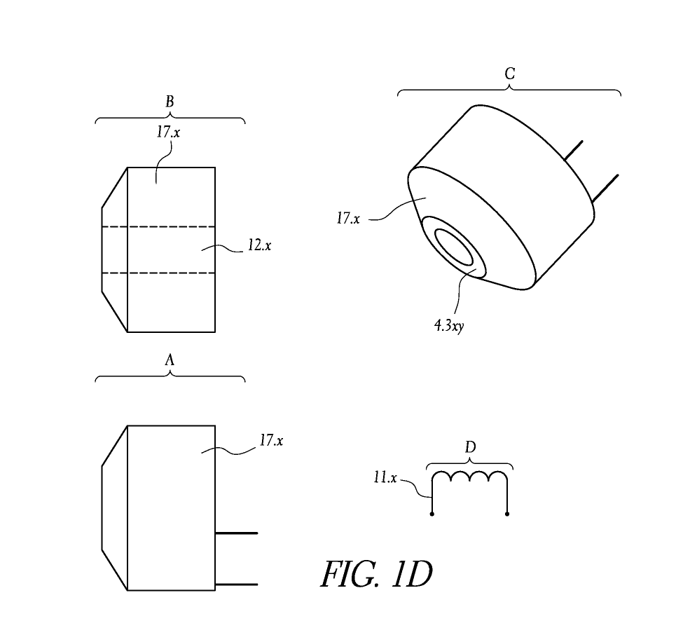

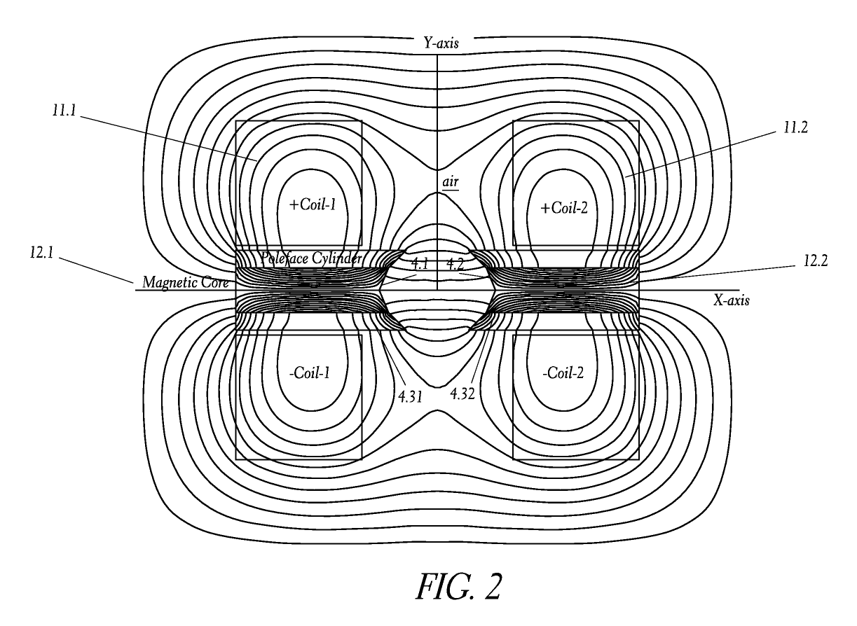

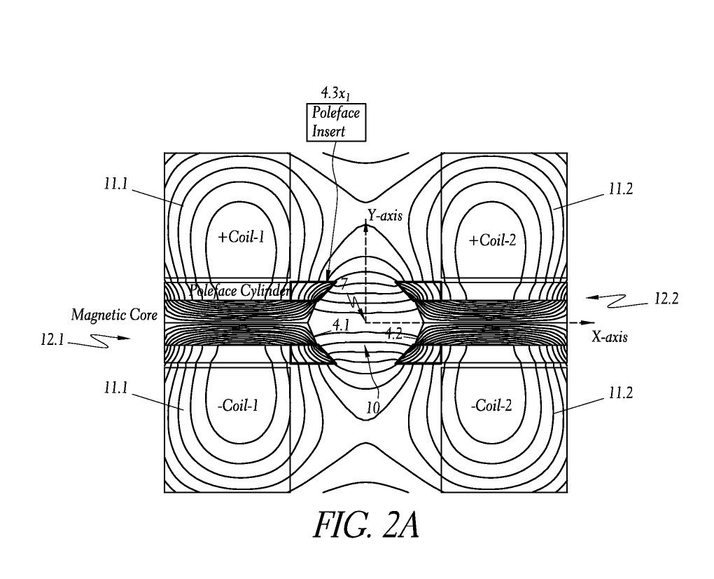

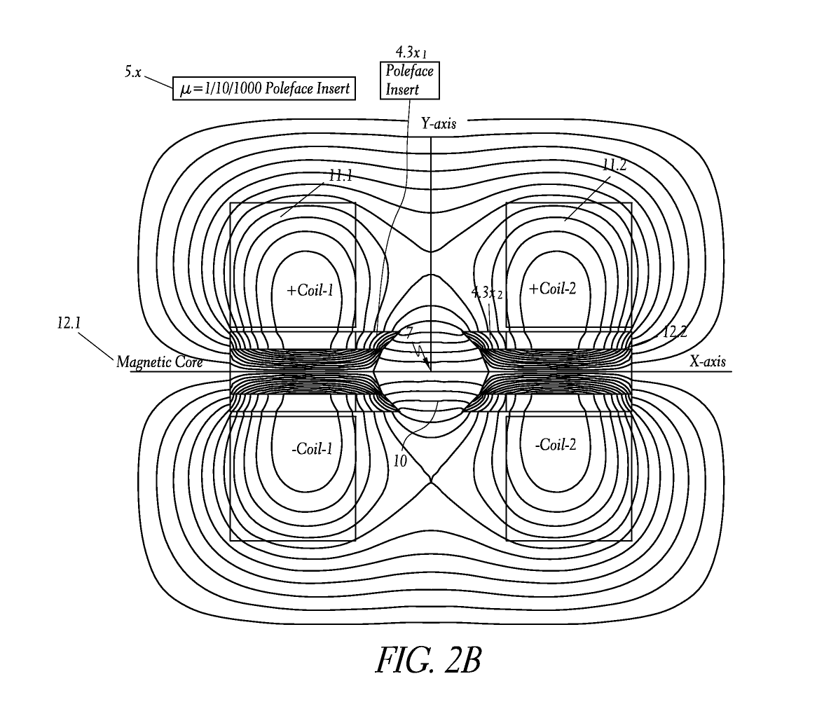

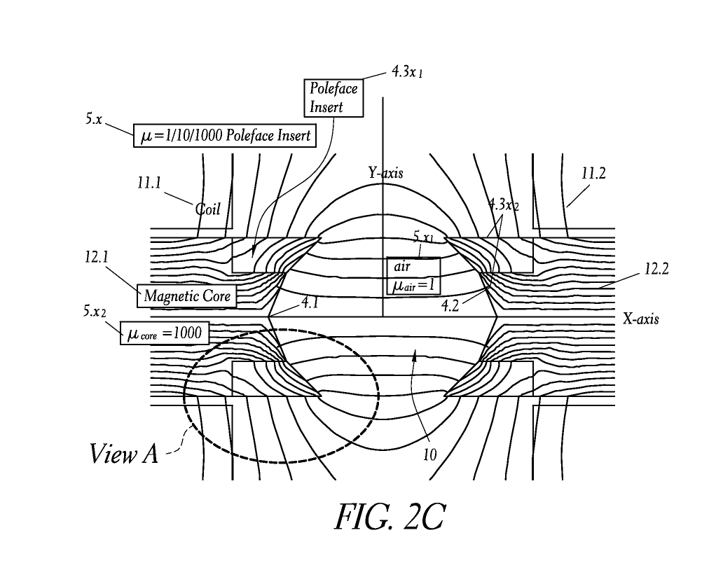

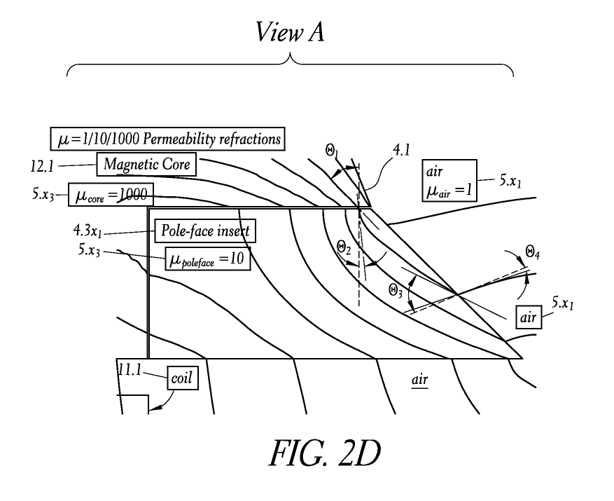

FIG. 2 shows one embodiment that provides further improvement of the flux-focusing and aperture control of the inner operating region 10. The poleface cylinder 4.31 and 4.32 are replaced with relatively narrower and smaller rings 4.3x₁ and 4.3x₂ around the poleface 4.1 and 4.2. The coils 11 are fitted with high permeability magnetic steel (μ>1000) under them, while the poleface 4.1 and 4.2 are divided into a high permeability (μ>1000) inner core 12.1 and 12.2 and a low permeability (μ>10) outer core 4.3x₁ and 4.3x₂. This division makes the poleface 4.1 and 4.2 behave as an anisotropic core material shaping the flux even more, so as to bend the magnetic flux line geometry toward the central operating region 10.

FIG. 2A is an orthographic depiction of the directional and flux density map whereby an equal or better performance characteristics of the “lensing” results, is presented by employing the segmented Poleface 4.1 and 4.2 rings are inserted 4.3x₁ and 4.3x₂ as indicated by the figure. This arrangement is modular and provides for insertion of different refraction indices based on demand or specificity of the task at hand. This method of combining the inserts 4.3x_{1 and} 4.3x₂ as low permeability ring arrangement improve the anisotropic geometry so as to “condense” the flux line density, while shifting the center of focus on demand. Experimental evaluation confirm better results of increase narrowing and focused flux through the magnetic lens 5.x₂, due to segmented or hybrid poleface material permeability.

FIG. 2B is a graphic representation of the directional and flux density map indicating equal or better performance with the segmented Poleface ring insertion with different permeability values (e.g., μ=1, μ=10, μ=1000). This arrangement of segmented hybrid permeability performs better as an aperture, narrowing and focusing the flux.

FIG. 2C is a graphic representation of the magnetic aperture 50, whereby a hybrid permeability of different materials is used to form the aperture to provide field focusing. The coil 11.1 is fitted with a magnetic core 12.1 with μ=1000, (ref. des.5.x3), the magnetic aperture 50, is augmented with a poleface insert with 4.3x₁μ=10 (ref. des.5.x₃). The effective area 10, has the permeability value μ=1, (Ref. des. 5.x₁), the resulting directional and flux density map is graphically shown in view “A”. The combination of poleface geometry, 4.3xy with different permeability values, 5.x_{y} is the reason by which the waveguide's lensing ability is improved.

The static solution derived from FIGS. 2C and 2D calculates as follows:

$${θ}_{1} = 45 ° {μ}_{1} = 1000 {μ}_{2} = 1 \\ tan {θ}_{2} = \frac{{μ}_{2}}{{μ}_{1} \cdot tan {θ}_{1}} thus {θ}_{2} = 0.65 ° \qquad 13 )$$

Thus, the magnetic flux again exits the poleface with close to perpendicular pointing from the concave-shaped surface into the operating region I0. The standing wavefront is altered based on combination of material permeability: 5.x_{y}[μ=1, μ=10, μ=1000.] and polefac geometry: 4.x.

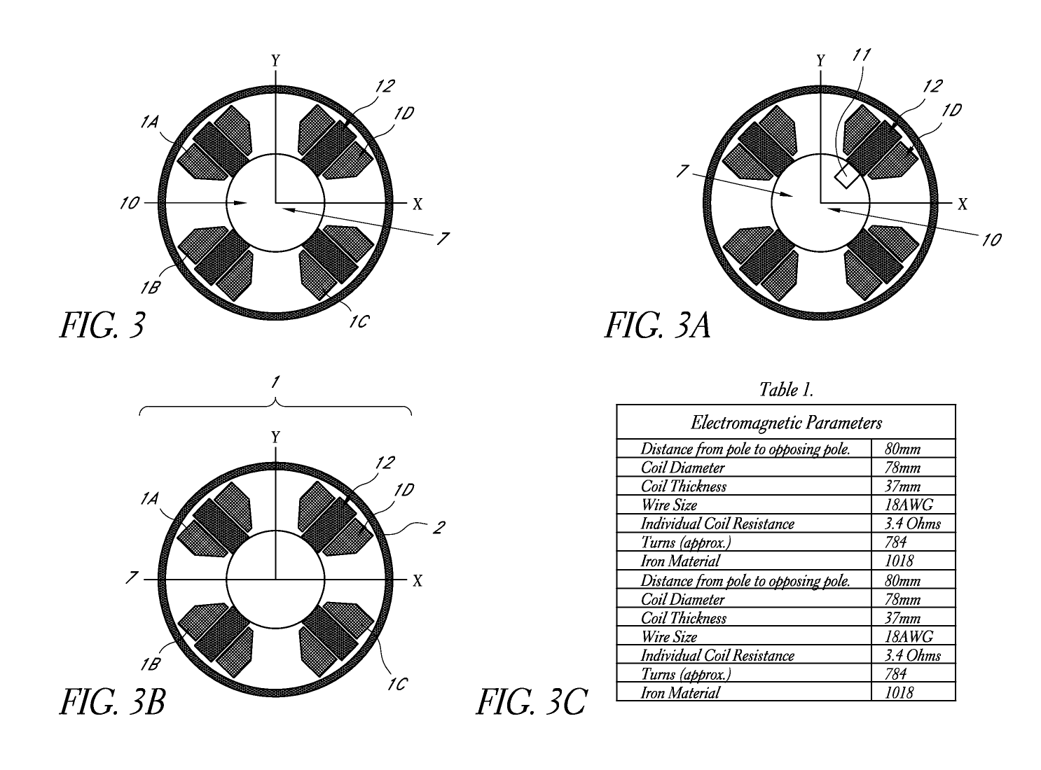

FIGS. 3 and 3A are descriptions of a scale model 1 and the rules of operations of the waveguide assembly. In one embodiment, the scale model 1 has an effective field region of 80 mm. One of ordinary skill in the art will realize that the effective field region can be scaled to any size smaller or larger than 80 mm, at least in part, by scaling the size of the magnet assemblies.

The scale model 1 is an embodiment of the waveguide 100, with counterpart coils with reference designator 17.1-17.8, and provides a containment ring for closing the magnetic circuit is designated by the scale model 1, using reference designator 2, is further defined by the waveguide 100, with reference designator 25.

The scale model 1 is constructed using four coils 1A, 1B, 1C, and 1D in the XY plane. The 2D configuration is supplemented with a flux return ring 2. The coil 1D is provided with an extendable iron core 3. The scale model 1 is approximately one-eighth the size of the full-scale waveguide 100, with 600 mm bore diameter. One of ordinary skill in the art will recognize that the full-scale waveguide 100 is not limited to the sizes listed here and can be constructed in any size as needed. The full size expansion is based on the four-coil XY plane (2D) scale-model 1, and a dual three plus three coil cluster XYZ (3D) 1.1. The results in tenns of geometry optimization as well as the topological transformation from 2D to 3D resulting in the contraction of eight coil configuration 100. The scale model I is fitted to the magnetic aperture 3a-d (polefaces). The pole pieces 3a-3d are used as a movable core so as to change the field's geometry, further used in magnetic shaping function, for the purpose of reducing coil size and power requirements while shifting the magnetic flux density's center. The optimization of the electromagnetic circuit is obtained as a geometrical expansion of the 2D scale model 1, further augmented by the topological transformation to the 3D model 1.1, which resulted in the forming the waveguide 100.

As shown by Table 1, by scaling the waveguide 100, it is possible to provide a 0.15-0.3 Tesla field density for torque control and a 1.6-3.0 Tesla/m field gradient for force control within the effective space 10. Using a 2.45 mm×10 mm size NbFe35 permanent magnet in the catheter tip 7, the scale model 1 (waveguide) is able to achieve 35 grams of force for catheter movement. The expansion of the scale model to a 3D eight coils 11, in the waveguide cluster generated a magnetic field in the center region 10, of the chamber 2. The waveguide is capable of exerting a torque on the catheter tip 7, in the desired direction, without an advancing force on the tip 7. This torque is used to bend and rotate the tip toward the selected direction. The magnetic field can also be configured to generate a relatively high field gradient in the center region 10, for exerting a moving force on the tip 7, (e.g., push-pull force), but without rotating torque on the tip.

The magnetic field of the scale model I can also generate a relatively high field gradient in the region 10 for exerting an orthogonal force on the tip 7 (sideways movement), without rotating torque on the tip. This is useful, for example, to align the tip at narrow forks of artery passages and for cleaning the sides of an artery.

The magnetic field within the scale model 1 can generate a mixed relatively high field strength and field gradient to push/pull and/or bend/rotate the tip 7, simultaneously. This is useful, for example, to guide the tip while it is moving in curved arteries.

The 80 mm scale model 1, shown in FIG. 3, is expanded using the scalability rules to a full scale waveguide 100, with 600 mm bore diameter by using the scaling equation:

$$\begin{matrix}AT ( r ) = {( 2 ⋆ \sqrt{3} )}^{\frac{ln ( r )}{ln ( 2 )}} & 14 ) \\ r = \frac{{D}_{scale}}{{D}_{demo}} = \frac{{D}_{scale} mm}{80 mm} & 15 )\end{matrix}$$

Scaling the demonstration unit 1, is fitted with poleface 11, mounted on the coils' core 3a-3d . The poleface (PF) 11 of the scale model 1, is employed by the waveguide 100, in forming the aperture that generate the specific geometry and flux density required in moving a magnetically tipped catheter. The PF 11 dimensions used follow the pole face diameter scaling multiplier.

$$PF ( r ) = {( 2 ⋆ \sqrt{2} )}^{\frac{ln ( r )}{ln ( 2 )}} \qquad 16 )$$

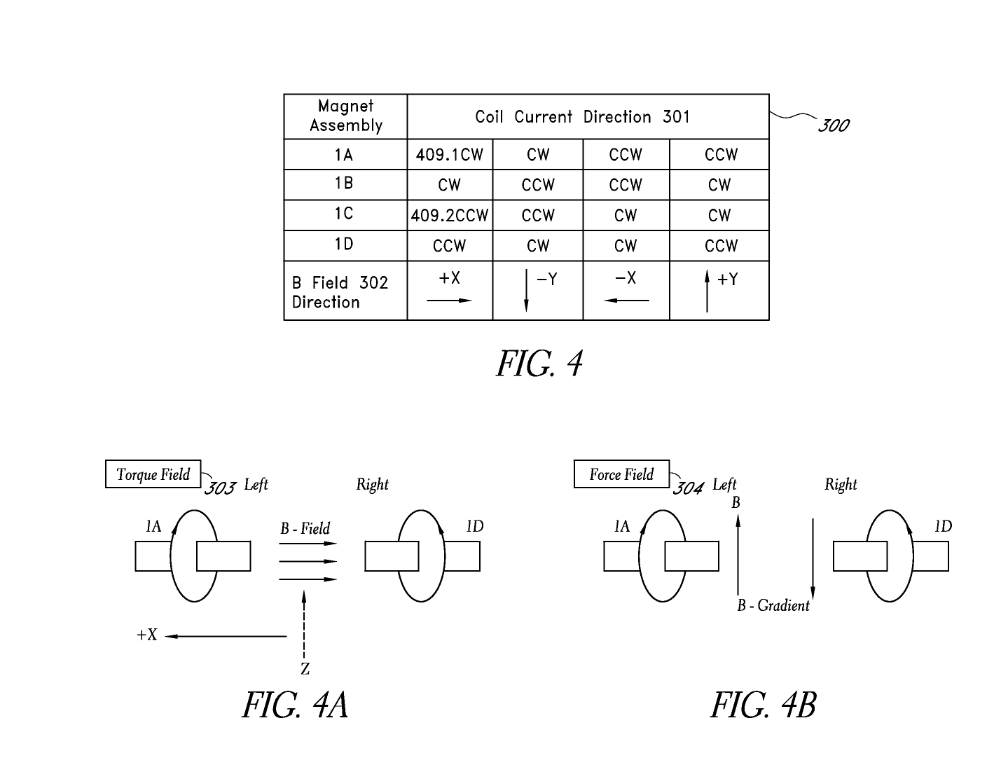

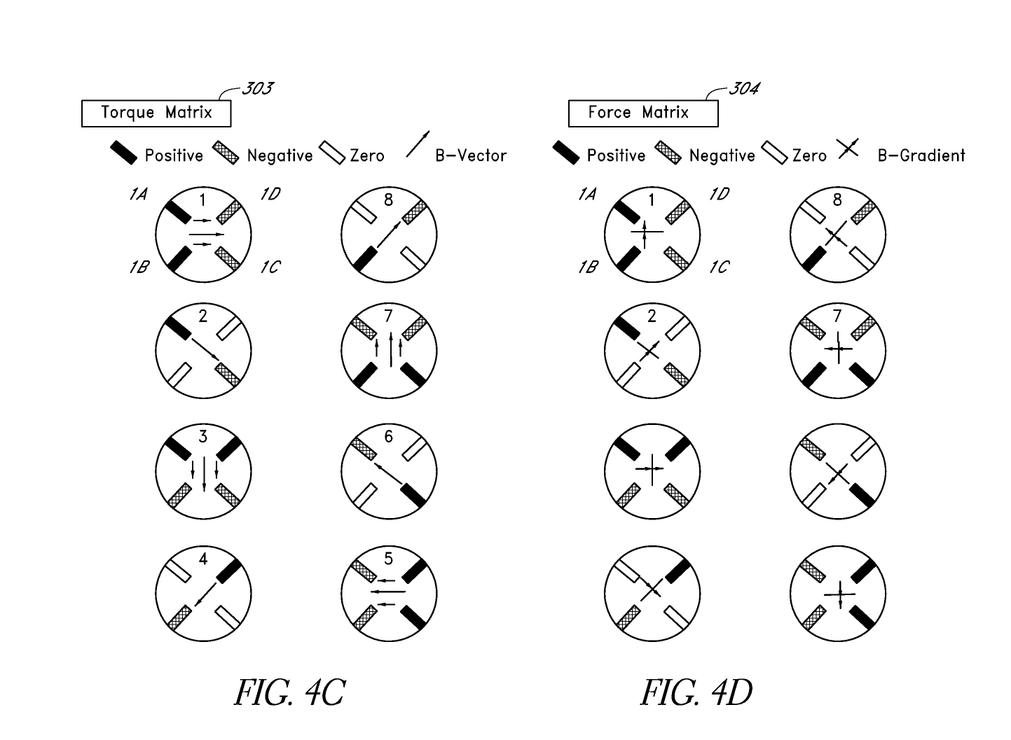

Forces on the catheter tip 7, permanent magnet (NbFe35) shown in FIG. 4A (2.45 mm radius and 10 mm length) is calculated as the force on a dipole in a magnetic field.

F_{M}=∇(B·M) 17)

Where M is the dipole magnetization vector and B is the field density vector around the dipole. Calculating B along axis S of the dipole, using the scalar derivative:

$${F}_{s} = M \cdot {A}_{m} \cdot {L}_{m} \cdot \frac{\partial B}{\partial s} \qquad 18 )$$

Where Aₘ is the magnetic cross section and Lₘ is its length.

$$\frac{\partial B}{\partial s} = 1.6 \frac{Tesla}{m} \\ M = 980 , 000 \frac{amp}{m} , {F}_{s} = 20.1 gram \qquad 19 )$$

For a maximum gradient,

$$\frac{\partial B}{\partial s} = 3 \frac{Tesla}{m}$$

In one embodiment of the magnetic aperture 50, the magnetic force field, generates

F_{S}=37 gram

The torque on the same size catheter tip 7, is calculated as the torque on the permanent magnet 7, in field B and is expressed:

Tₘ=M·B·Aₘ·Lₘ·sin(θ) 20)
and where θ is angle between the magnet axis and B.

Using an example for B=0.15 Tesla and an operating angle of θ=45°, gives:

Tₘ=0.013 Newton·m,

Hence the torque on a 10 mm arm with a 35 gram force is T_{35g}=0.0034 Newton·m.

Using B=0.15 Tesla yields a bending arm of 38 mm.

Using the scale factors in Equations (14) to (20), the waveguide 100 can be scaled so as to accomplish the desired tasks of control and navigation of the catheter tip 7, within the magnetic chamber 10. The example noted above demonstrated the improved performance of the scale model, while employing the inner cores 3a-3d by further fitting the cores with polefaces 11, so as to provide a mechanical shifting of the magnetic flux density center. By moving the cores and their associated polefaces, it is possible to form a specific geometry on demand. This feature is further exemplified by the drawings and accompanying descriptions.

FIGS. 4, 4A and 4B are summaries of the possible combinations of the waveguide 100, in generating the desired magnetic field and field gradient. By using the table, one can compute the matrix 300, so that the currents in the coils 1A, 1B, 1C and 1D are configured to generate the B field directions 302. The operation yields the desired magnetic force and force gradient as it is graphically illustrated by FIGS. 4A and 4B. The matrix illustrates the dependency of the current directions and magnitudes in the coils, whereby the center region can be set up for magnetic fields producing just torque 303, just force 304, or mixed torque and force 305. In the Torque Mode, four combinations of coil current directions in A, B, C, and D magnets produce an approximately uniform B field in the center region 10. The main B field vector directions (90° rotations) follow a rotational rule 300, shown in FIG. 4A.

FIGS. 4C and 4D show the rules that govern the performance of the waveguide I 00. The matrix 300, shown in FIG. 4, is annotated by indicating the field direction while the coil current is applied. The coil current polarities and magnitudes are set to produce the desired field directions for the torque and force fields. The torque field 303 generates combinations using an adjacent coil current direction such that the B-vector flows from core to core aiding each other. The coils 1A, 1B, etc., are viewed as if connected in series linked by a common magnetic field as shown in FIG. 4C. The force field 304, generating coil combinations uses an adjacent coil circulating their current such that they work against each other as shown in FIG. 4D. There are 64 combinations of positive and negative current flow polarities for the 8 coil design. The Scale model 1 is used as a baseline configuration. The four coils can have 16 combinations; half of them generate torque fields 303, the other half are force gradient configurations 304. Once the coil/polarity combinations are defined, they can be grouped into a set of matrixes according to above rules. Torque and force matrixes are extracted according to four coils and four coil groups associated with virtual 2D planes. The waveguide 100, is configured a spherical structure, whereby the eight coils are grouped as top coils: 1A_{T}, 1B_{T}, 1C_{T}, 1D_{T}, and a bottom coil group: 1A, 1B 1C and 1D, which form another group on a plane rotated 90° from the group above. Again there are 16 combinations for two/two sets of torque/force matrixes. The third group is formed as two triangular “side plane” combinations of 8 and 8 combinations for two/two sets of torque/force matrixes (mixed fields of torque and force magnetic field 305). Selecting the right combination of coils 1X and 1X_{T} and current polarities from each of these virtual planes is performed by the using a regulator 101, and the matrix algorithm 300, and by further applying the superposition rules that govern Maxwell vector field. The matrix algorithm 300 provides a coil/polarity combination set for any desired direction within the magnetic boundary. In case of possible multiple selection for the same mode and direction, the algorithm 300 selects a single combination based on possible combinations available for anticipated movement from AP to DP in the same direction and in accordance with the rules of optimal power setting.

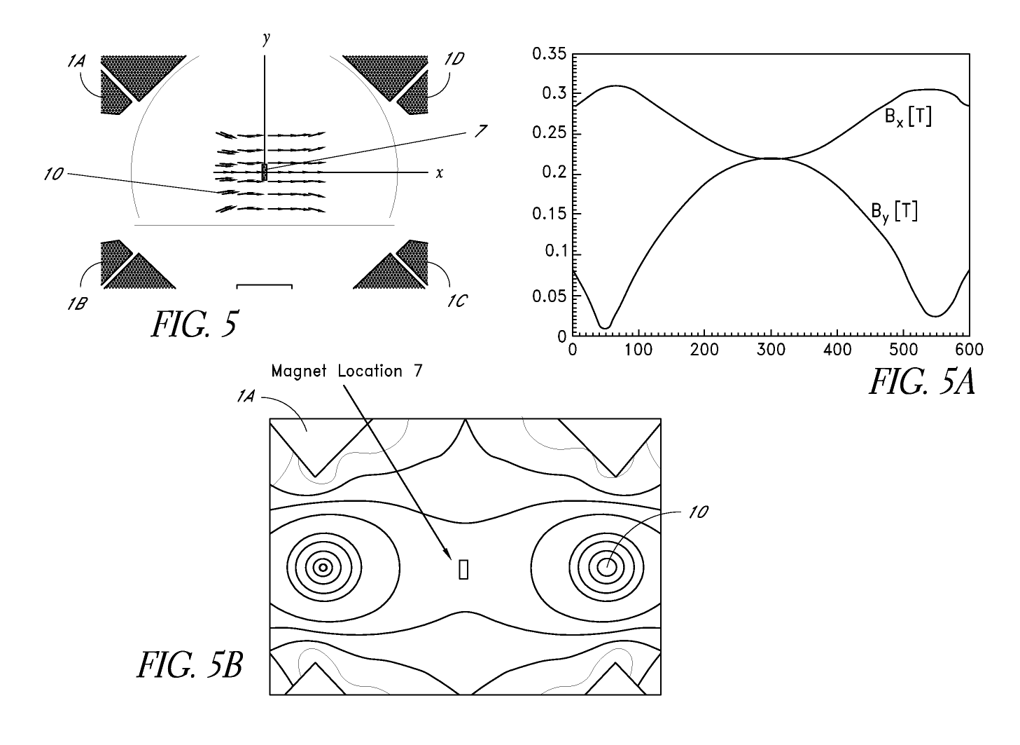

FIGS. 5, 5A and 5B illustrate the vector field plot of the B fields in a central region 10 of the waveguide 100. The set of examples with the figure, illustrate the ability of the waveguide to control and regulate the movement of a catheter with a magnetic element attached to the distal end to be push, pull and rotate on any axis relative to the magnetic wave front by using the waveguide 100 with its ability to form an optimal geometry relative to the tool (catheter) AP and its target DP. The apparatus 100, with its regulator 101 provides for movement of the medical tool in 3D space with 6 degrees of freedom while using the waveguide symmetry (topology), its EM radiators 17, and the improved anisotropy associated with its magnetic aperture 50. The following description illustrates the current configuration where the B-vector is parallel to the X-axis. The B vector is parallel to the +X axis and within the central region B is about 0.23 Tesla. The torque at a 45° angle between B and the magnet is 0.03 Newton meters. The example shown, by the case of +X indicate graphically an application of the coil current direction. The B field direction and the resultant position of the catheter tip 7, in the effective region 10, are shown. FIG. 5B shows the field intensity as a gradation from black to white on a scale of 0.02-0.4 Tesla. The electromagnetic circuit formed by coil's group 1A, 1B, 1C, and 1D applied in the effective region 10, and manipulated by the coil current direction and the B field direction generates the torque as well as force predicted by the algorithm 300, and with the formalism noted by equations (17) and (20).

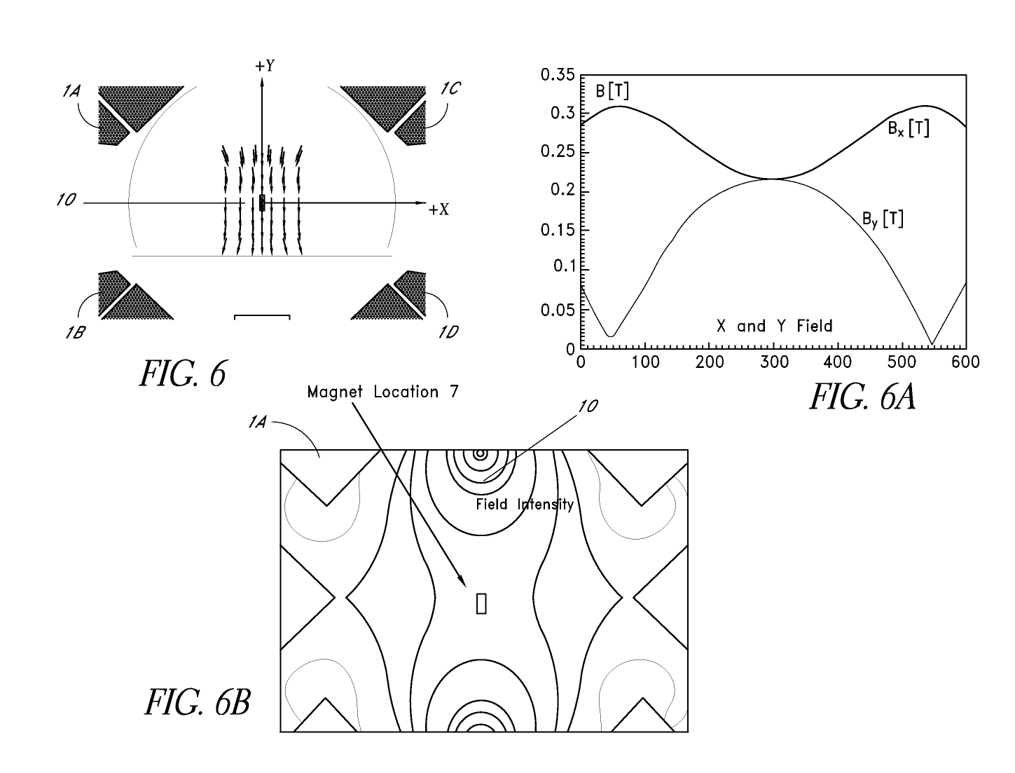

FIGS. 6, 6A and 6B further illustrate a case where the B vector is parallel to the −Y axis and within the central region/effective space 10, (±80 mm around the 600 mm bore diameter). B is about 0.23 Tesla, the torque is at a 45° angle between B and the magnet 7, is 0.03 Newton meters.

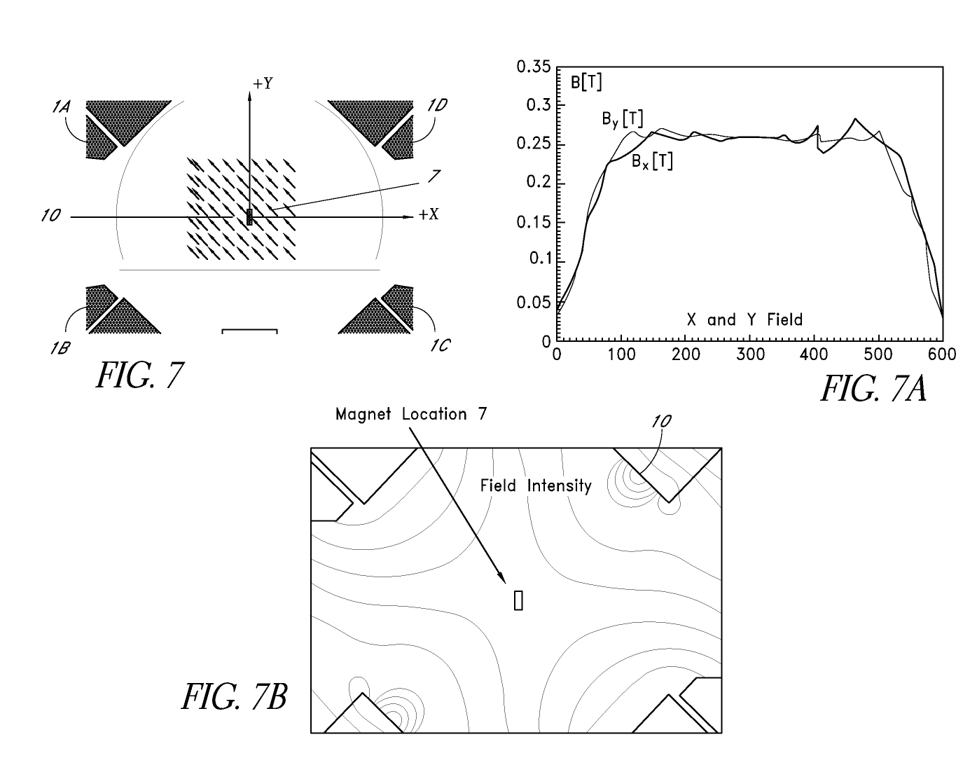

FIGS. 7, 7A and 7B illustrate the waveguide 100, and the matrix algorithm 300, where the boundary condition of the B vector (Torque mode 303), is pointing to the coil poleface 11, of coil 1A within the central region/effective space 10. The B value is set at about 0.195 Tesla. This 135° B vector direction is accomplished by setting the scale model 1, such that current in the coil 1A is directed as CCW, the current direction in the coil 1C is CW and the coil current of coils 1B and 1C are set at zero.

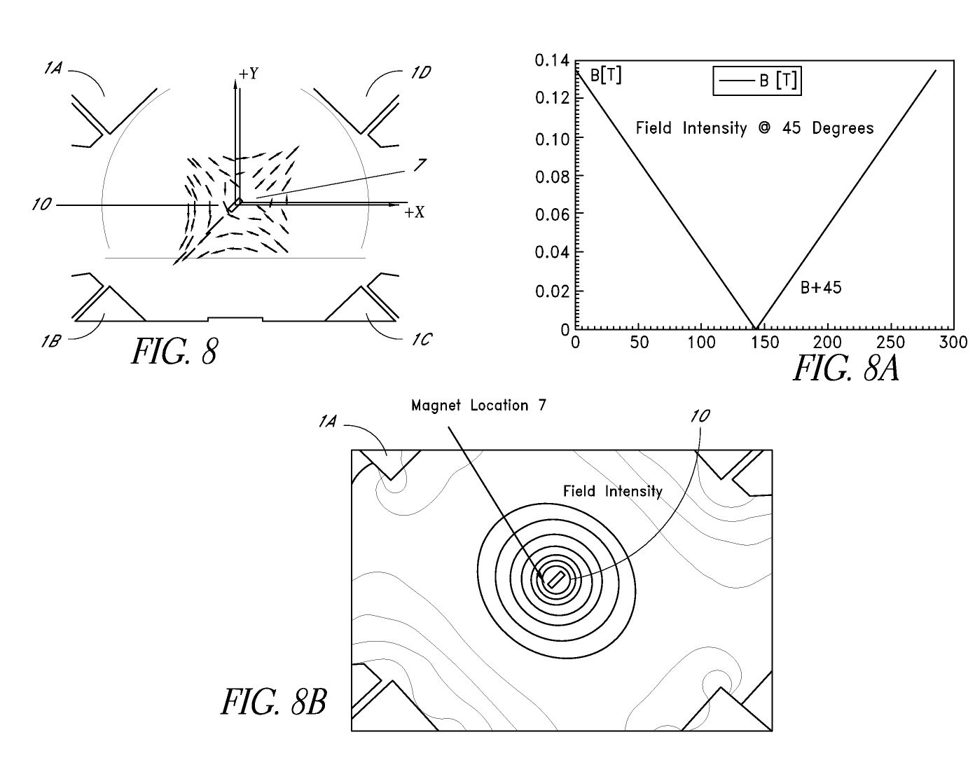

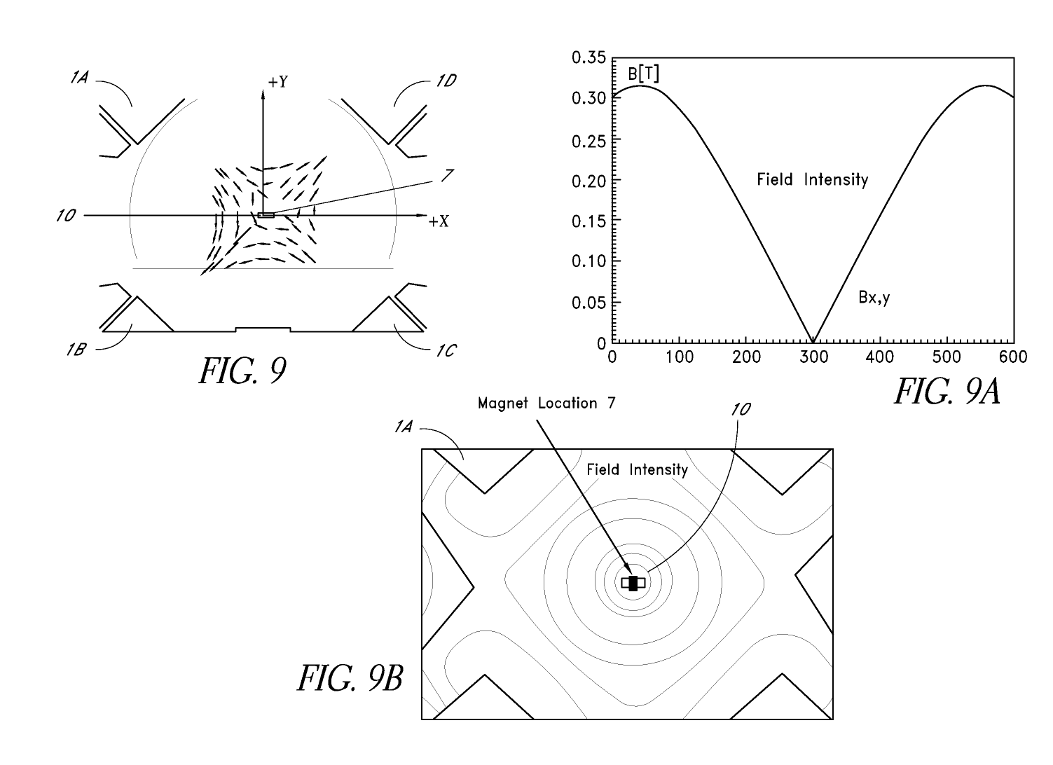

FIGS. 8, 8A and 8B illustrate the behavior of the model 1, in a force control 304, mode along the magnet axis with zero torque on the tip 7. In this case, coil 1D in a CCW current direction, coil 1B has CCW current, and coils 1A and 1C are set to zero current. The resultant force is 12 grams. FIGS. 9, 9A and 9B illustrate the force control mode 304, orthogonal to the magnet axis with a substantially zero torque on the catheter tip 7. In this case, the coil 1A is set at CW, and the coil 1B is set at CCW, the coil 1C at CW direction, and the coil 1D direction is CCW. The force is 22 grams.

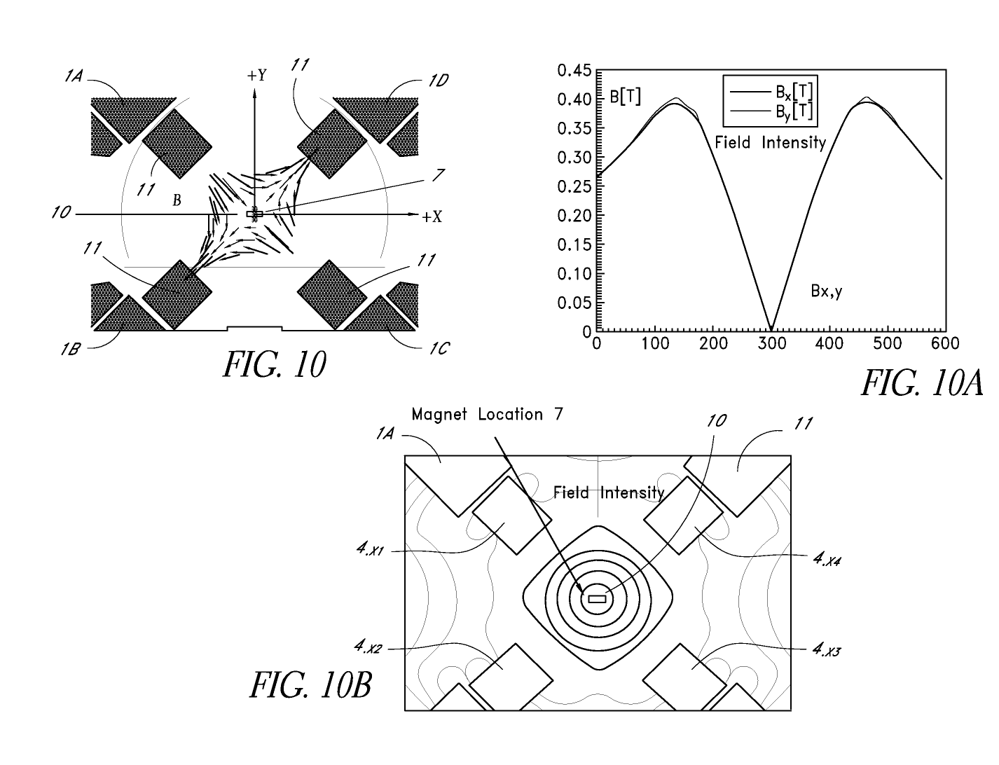

FIGS. 10, 10A and 10B illustrate the scale model 1, as it is set for the force control mode 304. This case demonstrates the use of the poleface 4.x with its core extension rod 12. The core extension 12, as it is influencing the magnetic field characteristics as disclosed above. FIGS. 10, 10A and 10B further depict the specific state of the force control 304. In the force control mode when the four cores are extended into the effective space 10, and where the coil 1A is set to CW, the coil 1B set to CCW, the coil 1C to CW and the coil iD is set to CCW. The resultant field geometry produces a force of 37 grams on the catheter tip 7 (see table 300 in FIG. 4 for detailed description of the calculus).

FIG. 11 is a graphic depiction of the four-coil formation 1A, 1B, 1C and 1D in the scale model 1, when the magnetic core extensions 11, with its poleface 4.x are deployed into the effective region 10. FIG. 11 further shows that by deploying the magnetic core extensions, the magnetic field is shaped. The figure also illustrates that the resulting magnetic field is relatively symmetrical and homogenous around the catheter tip 7.

FIG. 11A shows the core coil 1A with its core withdrawn, hence forming a new geometry configured to generate a shaped magnetic field for better control of the catheter movements in the effective space 10.

FIG. 11B is a graphic depiction of the shaped magnetic field when the core on coil 1D is retracted. The mechanical deployments of the cores 12, of the individual EM radiators is a simulation of the actual core with its magnetic aperture 50 used by the waveguide 100, and are an example of the notion of varying the permeability of the effective space 10, so as to form a shaped field on demand.

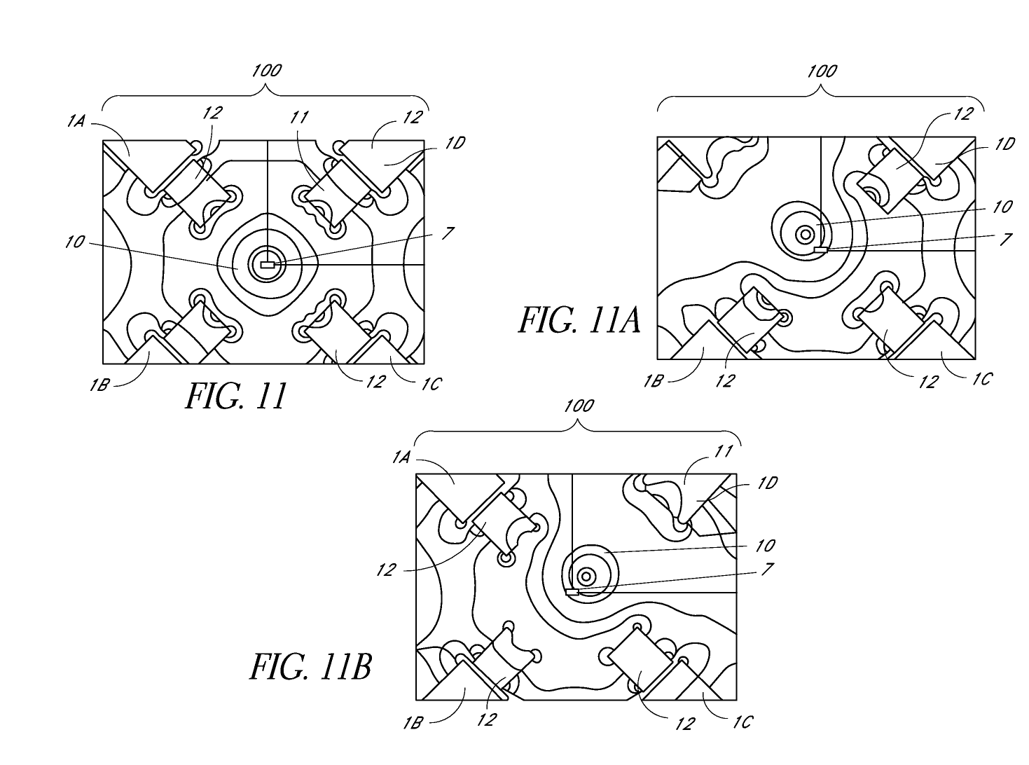

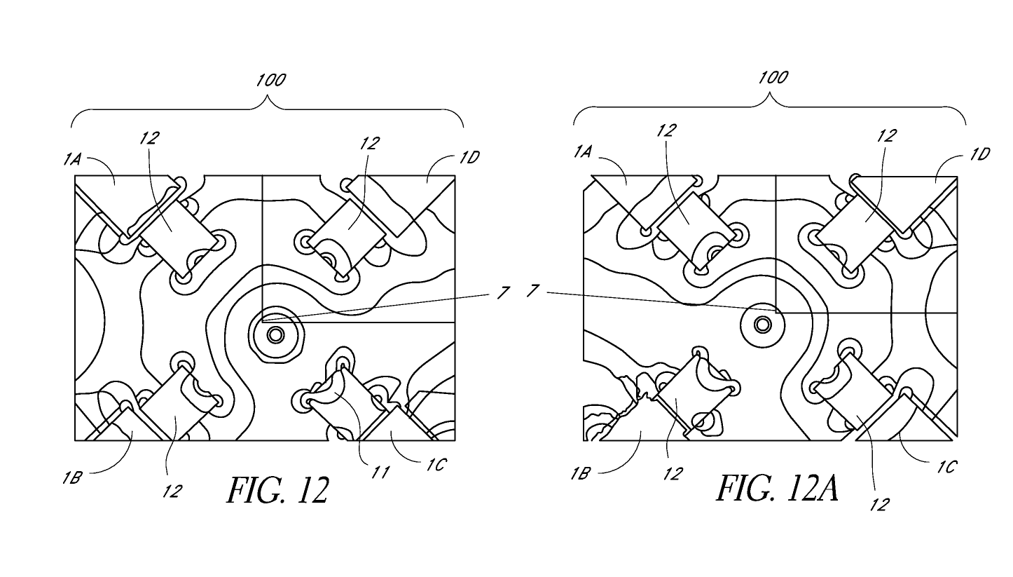

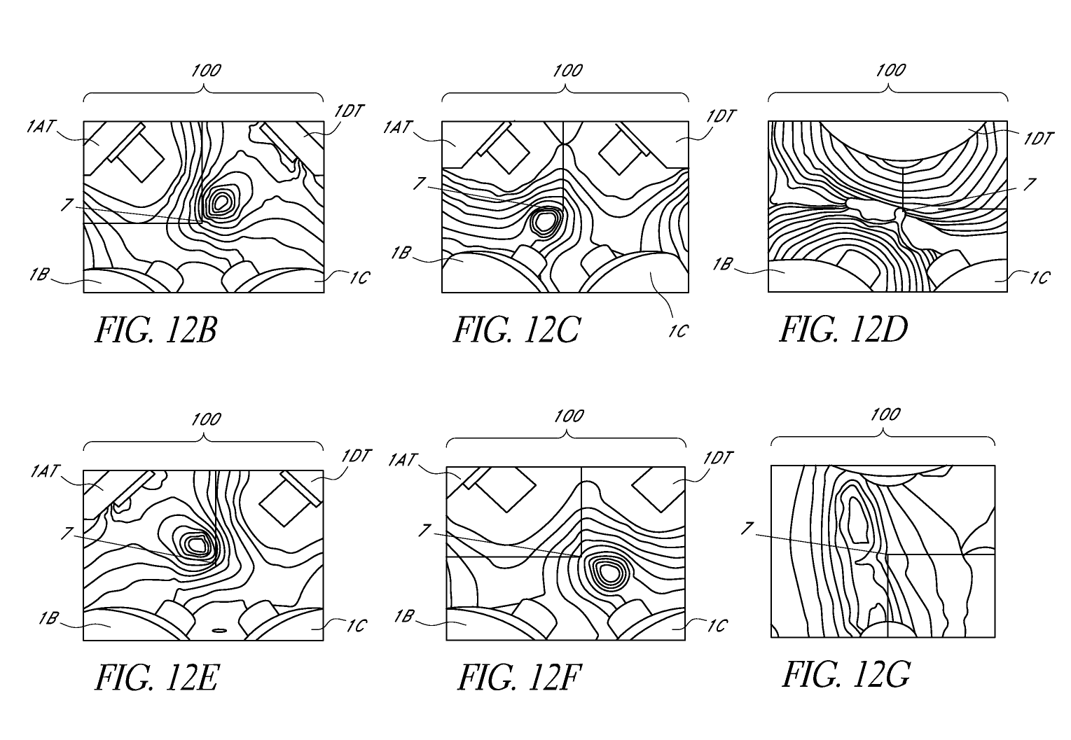

FIGS. 12 and 12A show the waveguide 100, and a configuration of the magnetic field geometry under the conditions where a generated field is formed by actuating and deploying the core extensions. As shown in FIG. 12, when the current on coil 1C is set at zero, the field has a similar geometry to that in FIGS. 11A and 11B, respectively. In the case of FIGS. 12 and 12A, the current of coils 1B and 1C are set at substantially zero, the magnetic extension core 11 and its associated poleface 4.x is varying the deployment distance, hence varying the field geometry relative to its respective position. The shaped magnetic field using the variable-length extension cores allows the creation of effective magnetic field geometry for control and navigation of the catheter tip 7 within the effective space 10.

FIGS. 12B, 12C, 12D, 12E, 12F and 12G are graphic depictions of various states of the waveguide performance as a combination of direction as well as power intensities is demonstrated. The algorithm 300 in the torque mode 303, force mode 304 and mixed fields 305, are demonstrated. The waveguide 100 wherein a combination of the cores 12 and current control are used in shaping the magnetic field characteristics. The resultant magnetic field geometry allows the waveguide 100, to shape the magnetic field by varying the magnetic circuit characteristics and by extending and/or retracting the cores while varying the PWM, (duty cycle), on the power supply 102, and amplifier 103, respectively. The cores are identified as 1A_{T} through 1D_{T} respectively.

FIG. 12B shows a condition wherein the core 1A_{T} is deployed while core 1D_{T} is retracted. The magnetic field is measured along the XZ plane.

FIG. 12C shows the cores 1A_{T} and 1D_{T} fully extended. The magnet current is set at 1%.

FIG. 12D shows the coils 1B and 1C where current control is set at 1% along the YZ plane.

FIG. 12E shows a condition wherein core 1A_{T} is retracted. The forces are shown on the XZ plane.

FIG. 12F shows the coils 1B and 1C at a current of 1% on the XZ plane where the geometry accommodates the catheter tip 7, control as shown.

FIG. 12G is a graphic representation of coil currents 1A and 1B at +100%, coils 1C and 1D are at −100% and 1% respectively along the XY plane.

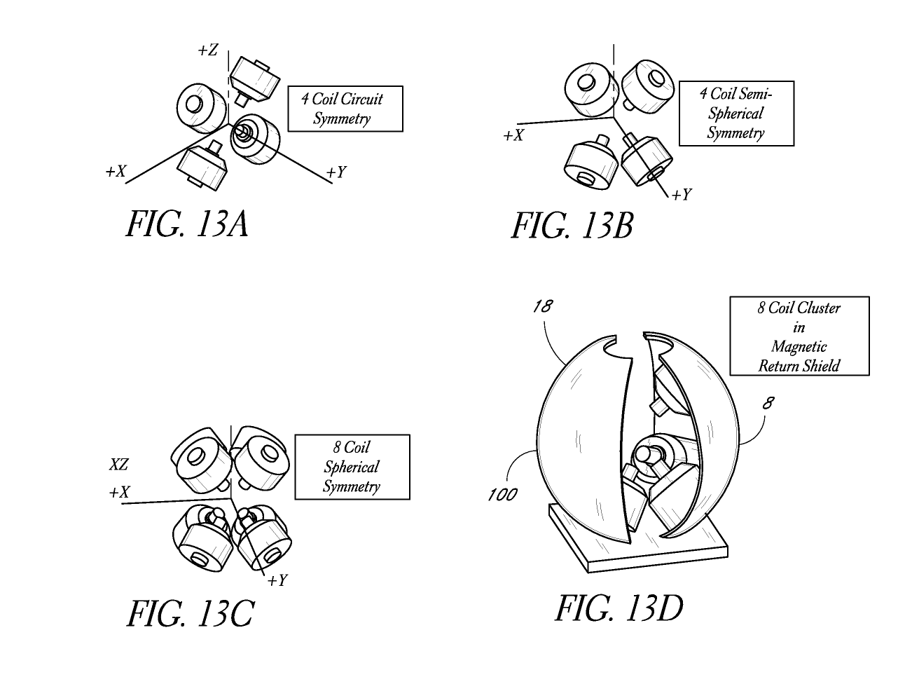

FIGS. 13A, 13B, 13C, and 13D are isometric representations of the waveguide 100 topology, whereby the use of the scaling equations as applied to the scale model 1, and by further expanding the scale model 2D four-coil geometry (80 mm) to the 3D full scale eight coils spherical geometry. The scaling rules noted above and the magnetic force equations are used in combination with coil current polarity and polarity rotation to generate the desired magnetic field in the waveguide 100. The topological transformation provides for the creation of base symmetry whereby a linear application of vector field calculus is preserved within the effective space 10 of the waveguide. The symmetry of the waveguide 100 allows the regulator to perform a linear translation and rotation, including elevation of the manifold.

FIG. 13C is an isometric representation of the first order expansion from the 2D (80 mm) scale model 1, to a topologically symmetrical four coil cluster. The third iteration of FIG. 13C wherein the four coils shown in FIG. 13B are mirror imaged on the XY plane to produce an eight coil spherical symmetry.

FIG. 13D is an isometric representation of the second order expansion of FIG. 13C to four coils rotated 45° in the +Y direction on a surface of a sphere to give a four coil semi-spherical symmetry cluster.

FIG. 13D further describes the need to shield the waveguide 100, wherein the configuration coil cluster shown in FIG. 13C is encased with parabolic flux return antennas 18, and is defined by its transformation encasement of the eight coil cluster into the YZ symmetrical magnetic return shield. The shield provided by the parabolic antenna collect the stray magnetic fields emanating from the EM radiators 17.1-17.8 and further improves the efficiency of the waveguide 100.

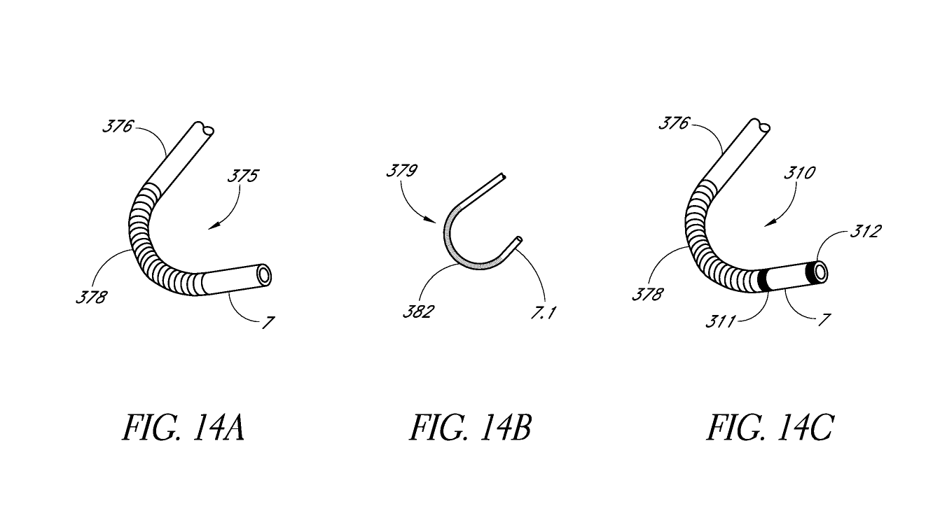

FIGS. 14A, 14B and 14C are orthographic representations of the medical tool(s) such as a catheter, fitted with a permanent magnet, or an articulated set of permanent magnets in the distal end of the tool. The catheter assembly 375 is a tubular tool that includes a catheter body 376, which extends into a flexible section 378 that possesses sufficient flexibility for allowing a relatively more rigid responsive tip 7, to be steered through the patient's body vascular or body's orifice.

In one embodiment, the magnetic catheter assembly 375, in combination with the waveguide apparatus 100, reduces or eliminates the need for the plethora of shapes normally needed to perform diagnostic and therapeutic procedures. During a conventional catheterization procedure, the surgeon often encounters difficulty in guiding the conventional catheter to the desired position, since the process is manual and relies on manual dexterity to maneuver the catheter through a tortuous path of, for example, the cardiovascular system. Thus, a plethora of catheters in varying sizes and shapes are to be made available to the surgeon in order to assist him/her in the task, since such tasks require different bends in different situations due to natural anatomical variations within and between patients.

By using the waveguide 100, and while manipulating the tool distal magnetic element, only a single catheter is needed for most, if not all geometries associated with the vascular or the heart chambers. The catheterization procedure is now achieved with the help of the waveguide 100, which guides the magnetic catheter and/or guidewire assembly, 375 and 379, to the desired position (DP), within the patient's body 390 as dictated by the surgeon's manipulation of the virtual tip 905. The magnetic catheter and guidewire assembly 375, 379 (i.e., the magnetic tip 7, can be attracted or repelled by the electromagnets of the waveguide apparatus 100.) provides the flexibility needed to overcome tortuous paths, since the waveguide 100 overcomes most, if not all the physical limitations faced by the surgeon while attempting to manually advance the catheter tip 7, through the patient's body.

In one embodiment, the catheter tip 7, includes a guidewire assembly 379, a guidewire body 380 and a tip 381 response to magnetic fields. The Tip 377 steered around sharp bends so as to navigate a torturous path. The responsive tips 7 of both the catheter assembly 375 and the guidewire assembly 379, respectively, include magnetic elements such as permanent magnets. The tips 7 and 381 include permanent magnets that respond to the external flux generated by the waveguide's electromagnets.

In one embodiment, the responsive tip 7 of the catheter assembly 375 is tubular, and the responsive tip is a solid cylinder. The responsive tip 7 of the catheter assembly 375 is a dipole with longitudinal polar orientation created by the two ends of the magnetic element positioned longitudinally within it. The responsive tip 7 of the guidewire assembly 379 is a dipole with longitudinal polar orientation created by two ends of the magnetic element 7 positioned longitudinally within it. These longitudinal dipoles allow the manipulation of both responsive tip 7 and with the waveguide 100, as its electromagnet radiators 17.x, and will act on the tip 7 and “drag” them in unison to a desired position as dictated by the operator.

In one embodiment, a high performance permanent magnet is used in forming the distal end of the tool so as to simultaneously have high remanence Mᵣ, high Curie temperature T_{c} and strong uniaxial anisotropy. Further, properties of the permanent magnate 7 is its coercive field H_{c}, (defined as the reverse field required to reduce the magnetization to zero), and where the (BH)ₘₐₓ is inversely proportional to the volume of permanent magnet material needed to produce a magnetic field in a given volume of space.

In one embodiment, a permanent magnet such as Nd₂Fe₁₄B is used in forming the distal end of the tool, providing for a saturation magnetization of about 16 kG.

FIG. 14C describes a possible formation of a catheter tip 310, whereby the permanent magnet 7, is supplemented with additional set of small beads. The magnet 7 and the beads 378 are fabricated using magnetic materials and chemical composition having at least two different H_{c} values to produce a universal joint. The magnetic field B emanating from the waveguide's EM radiators 17.x is applied uniformly onto the axial magnetization of the magnetic tip 7 and 378. The two elements forming the assembly, with distinctly different H_{c} values will act on each other as a mechanical joint (a cantilever action of the element 7, pivoting on arm of 378 due to the field uniform, emanating from the EM generators 17.x on the axis of magnetization of element 7 and 378 can be unpatented by using different combinations of geometry, mass, coercivity and permeability of the assembly; permanent magnet 7, and its secondary element 378, by further forming a magnetically coupled joint. The two different H_{c} values having properties that are “elastic” or “plastic” will responds to the magnetic field B in a fashion of simulating an action such as cantilevered beam, and the deformation will results in an angular displacement value associated with the H_{c} values difference. When the Force F1 (generated by the B field) is removed, the cantilevered moment of inertia will recover and return to the position of its natural magnetization axis.

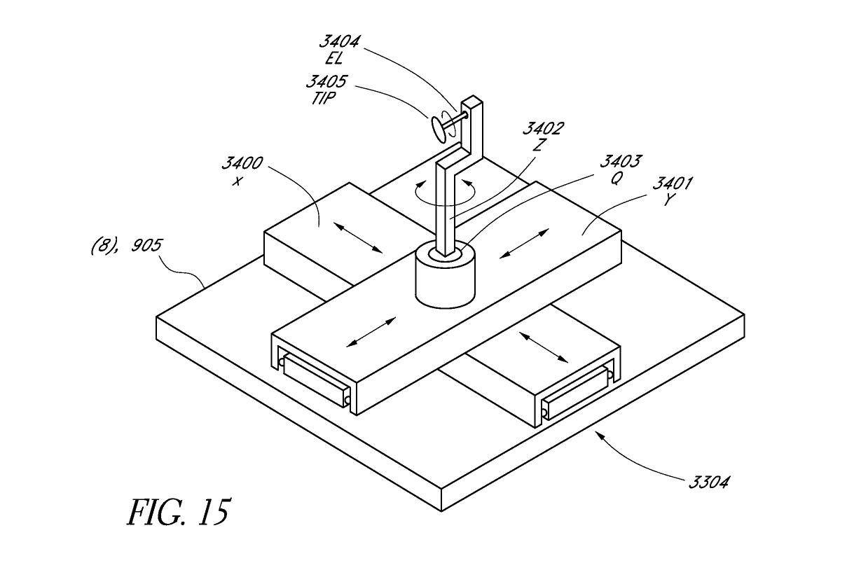

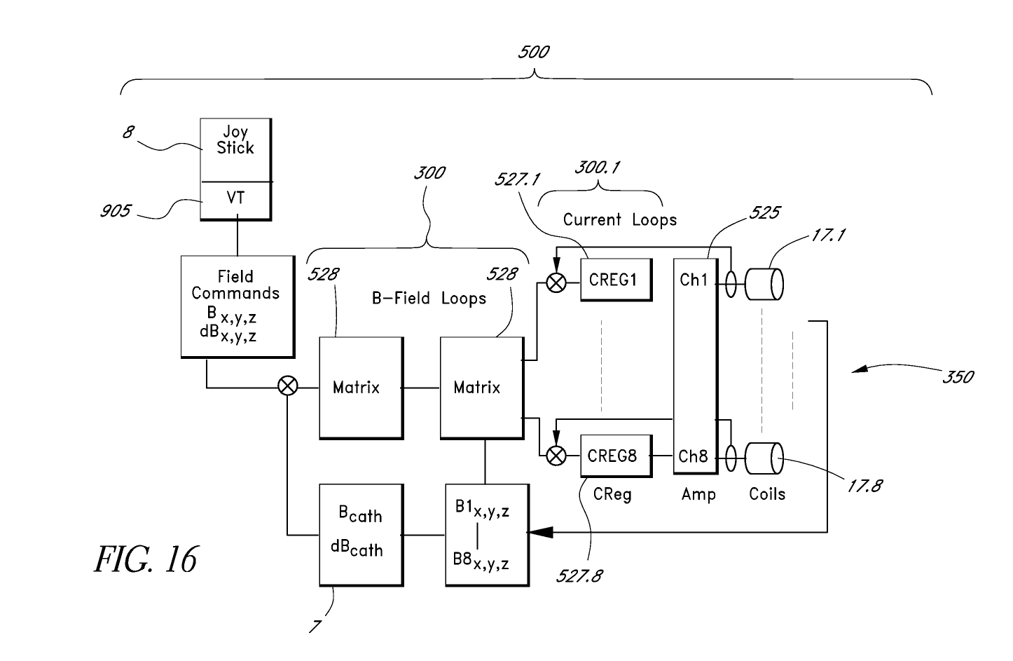

FIG. 15 is a perspective view showing one embodiment of the Virtual Tip user input device 905. The Virtual Tip 905 is a multi-axis joystick-type device 8, which allows ,the surgeon to provide inputs to control the position, orientation, and rotation of the catheter tip 7, within the waveguide 100 chamber.

In one embodiment, the Virtual Tip 905 includes an X input 3400, a Y input 3401, Z Input 3402, and a phi rotation input 3403 for controlling the position of the catheter tip. The Virtual Tip 905 further includes a tip rotation 3405 and a tip elevation input 3404. As described above, the surgeon manipulates the Virtual Tip 905 and the Virtual Tip 905 communicates the surgeon's movements to the controller 500. The controller 500 then generates currents 300.1 in the coils (EM generator 17.x ), to effect motion of actual catheter tip 7, to cause actual catheter tip 7 to follow the motions of the Virtual Tip 905. In one embodiment, the Virtual Tip 905, includes various motors and/or actuators (e.g., permanent-magnet motors/actuators, stepper motors, linear motors, piezoelectric motors, linear actuators, etc.) to provide force feedback 528, to the operator to provide tactile indications that the catheter tip 7, has encountered an obstruction of obstacle.

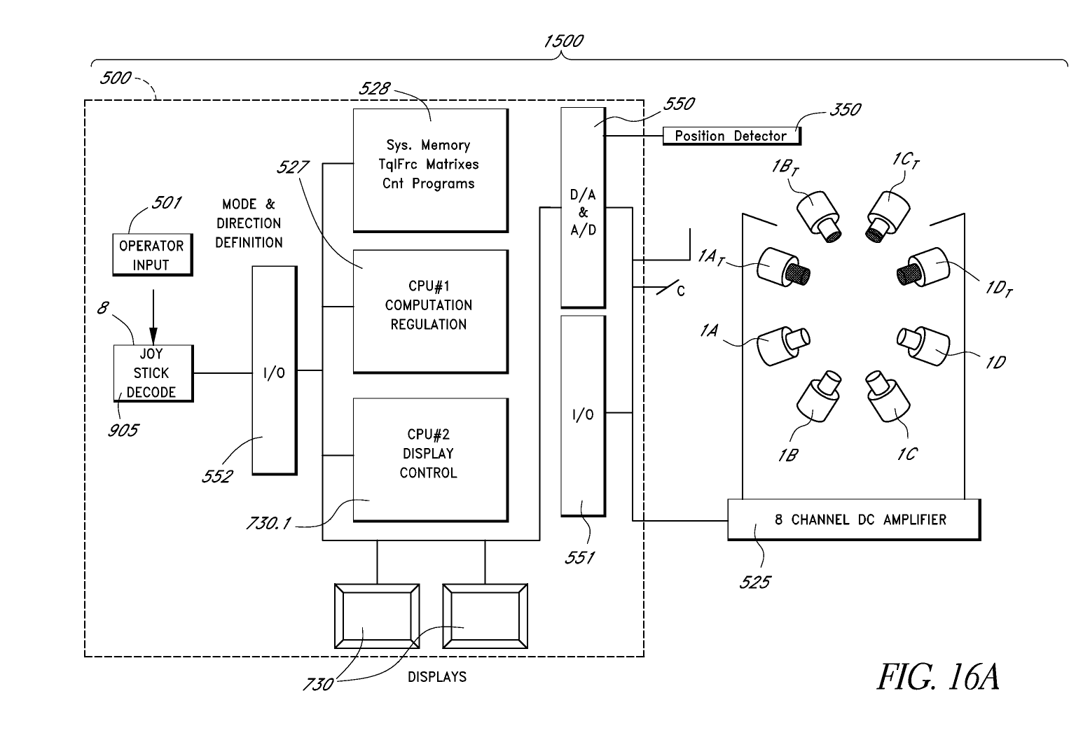

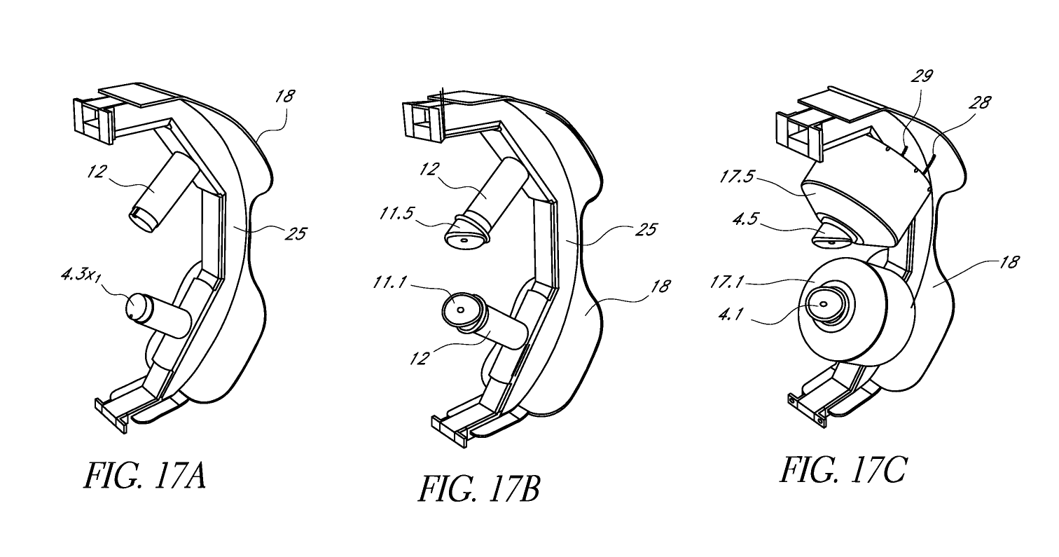

FIGS. 16 and 16A illustrate the field regulator loop 300, whereby a position detection sensor output 350 (such as Hall effect sensor, Radar, Impedance detector, 4D Ultrasonic probe and others imaging modalities and as detailed by Shachar U.S. Pat. No. 7,280,863) is used in establishing the AP coordinate set (3 vectors set for position and 3 vectors set for orientation). A detailed description of the method and apparatus for establishing the dynamics of AP of catheter tip is further described in US applications and international application Ser. No. 12/099,079, Apparatus and Method for Lorentz-Active Sheath Display and Control of Surgical Tools, PCT/US2009/039659, Apparatus and Method for Lorentz-Active Sheath Display and Control of Surgical Tools, Ser. No. 12/113,804, Method and Apparatus for Creating a High Resolution Map of the Electrical and Mechanical Properties of the Heart, PCT/US2009/040242, Method and Apparatus for Creating a High Resolution Map of the Electrical and Mechanical Properties of the Heart, hereby incorporated by reference. One embodiment described herein includes a closed loop control system for controlling the waveguide 100 and guiding the catheter tip. FIG. 16 further shows the EM generator 17.x , interface joystick 8, and its virtual tip 905, where the user commands are initiated. In one embodiment, movement of the catheter tip 7, is initiated as a field having a vector with components Bx, By, and Bz, for torque control 304, and a vector Bx, By, Bz for force control 303, are computed using algorithm 300. The B-field loop with its functional units, include a regulator 901, Position detector sensor 350, means to measure the B and dB fields. Computation regulators 527 calculate position, desired position (DP) change and the desired field and field gradients. The coil current 17.x is set and the catheter tip 7, position is changed from actual position (AP) to desired position (DP). 102141 In one embodiment, the movement of the catheter tip 7, is seen in real time by the operator 500 while observing the display 730. The “fire” push-button on the (JS) 8, selects torque or force modes for “rotate” or “move” commands. The magnitude and direction of the torque and force are determined by user inputs to the JS 8.

In one embodiment, the system sets the maximum torque and force by limiting the maximum currents.

In one embodiment, catheter movement is stopped by releasing the JS 8. The fields are held constant by “freezing” the last coil 17.x , current values. The magnetic tip 7, is held in this position until the JS 8, is advanced again. The computer 527 also memorizes the last set of current values. The memorized coil matrix sequences along the catheter movement creating a computational track-record useful for the computer to decide matrix combinations for the next anticipated movements.

In one embodiment, the magnetic. field is sensed position detection scheme 350. The position detector 350, provides the Bx, By, and Bz components of the field sufficient to describe the 2D boundary conditions numerically. The measurements are used to calculate B magnitude and angle for each 2D plane. From the fixed physical relationship between the plane centers, the field can be calculated for the catheter 7.

In one embodiment, the position detector 350 produces analog outputs, one for each component, for the A/D converter 550. This data is used to compute the superimposed fields in the 3D region of the catheter 7 (effective space 10).

Another embodiment of the waveguide regulator 500 uses close loop control wherein the biasing of the field is performed without the visual man-in-the-loop joystick 8, feedback, but through position control and a digital “road-map” based on a pre-operative data generated by digital coordinate derived from imaging techniques such as the MRI, PET Scan, etc. The digital road map allows the waveguide regulator 500, and the position detector 350, to perform an autonomous movement from the AP to DP based on closed loop control.

Field regulation matrices 303 and 304, are based on providing the coil current control loops 300.1 used in the manual navigation system within the field regulating loop 528, as a minor loop, and to be a correction and/or supervisory authority over machine operation. Control of B-field loops is defined by the joystick 8, and the virtual tip (VT) 905, and its associated field commands 300.

FIGS. 16 and 16A as noted by system 1500, further indicates the ability of the field regulation 300, to perform the tasks of moving the catheter tip 7, from AP to DP with accuracy necessary for delivering a medical tool in vivo. The field regulator 300 receives a command signal field 303, 304, from the position detector 350, and the JS 8, new position DP data from the computation unit 300, which generates a Bx, By, Bz vector for torque control, and the dBx, dBy, dBz vector gradient for force control. This position computational value identified in FIG. 16 allows the regulator 500, to receive two sets of field values for comparison.

The present value (AP) of Bcath and dBcath 300.1, acting on the catheter tip 7, are calculated from the position detector 350, outputs B x, y, z. The new field values for the desired position (DP) Bx, By, Bz 303, and dBx, dBy, dBz 304, to advance the catheter tip 7, are generated in the waveguide regulator 500. The difference is translated to the Matrix block 528 for setting the coil currents 300.1, and polarities as it is. graphically shown by FIGS. 4C and 4D.

In one embodiment, the matrix 528, issues the current reference signals to the eight regulators CREG 527.1-527.8 individually based on the needs of the path translation or rotation from AP to DP. The regulators 500 drive the eight-channel power amplifier 525, to obtain the desired coil currents.

In one embodiment, the torque on a permanent magnet 7, in field B is as noted by equation (20) above:

T=M·B·Aₘ·Lₘ·sin(θ)

Where M is the dipole magnetization vector, and B is the field density vector around the dipole.

Aₘ is the magnet cross section, and Lₘ is its length. For B—0.15 Tesla the calculated bending arm is L_{bend}=38 mm. Assuming B is measured with 1% error, Tₘ will have a 1% error.

Therefore, the position error due to measuring error of 1% is:

$${L}_{error} = \frac{{L}_{bend}}{100} = 0.38 mm or 0.015 inch .$$

FIGS. 17A, 17B and 17C are orthographic representations of the waveguide 100, mechanical elements forming the waveguide chamber. The architecture of the waveguide and its metric dimensions are the results of the topological transformations and scalability rules noted above. The materials with the specific permeability are subject to the derivation guided by the need to form an homogenous magnetic fields within the effective space without anisotropic variations within the effective space. Further considerations associated with symmetry of the EM radiators wavefront characteristics were incorporated in accordance with the design construct such as ferro-magnetic refraction, isotropic and anisotropic radiation, linear superposition principles and field intensities within the effective space. The waveguide assembly includes four right and left symmetrical structure, whereby a magnetic conductor arm 25, is formed to its shape, using a magnet steel A848 near pure iron with permeability “C” chemical composition. In one embodiment the arm 25, serve as a conductor to collected stray magnetic fields radiating beyond the effective space 10, and improve the efficiency of the waveguide as it act as a secondary containments for the energy when the EM radiators 17.1-17.8, are switching from one state to its required mode, (Based on the regulator demands due to AP-DP transition path), by varying the current of coils 17.1-17.8, the generated EM fields are defined by B value in percent (%) by employing the following expression:

$${B}_{\%} = 100 \cdot sin [ \frac{{I}_{A}}{100} ] \qquad 21 )$$

Where IA varies from 0 to 100. The regulator 500, computes the rotational angle according to the following equation:

$$θ = - \frac{1}{2} {cos}^{- 1} [ \frac{{I}_{D}}{{I}_{A}} ] \qquad 22 )$$

Where I_{A} and I_{D}, for example, are, for example, coils 17.1 and 17.5 currents and are switched so as to supply the needed energy to move or rotate the catheter tip 7, from its AP 5 state to DP 6 state. The rotational procedure 303, uses the regulator 500, which controls the eight coils to rise to full duty cycle together according to the L/R time constant, and lines up to +X at zero degree phase. The regulator controls the coils 17.1-17.8 to its zero duty cycle. The phase rotates to −45° while the field strength remains constant. The regulator commands current of coils 17.1-17.8, to reverse. The phase angle rotates to −90° while the field strength remains constant. These procedures generate a surplus energy which the magnetic conductors 25.1-25.4 channel and partially absorbed, during the transitory state of the waveguide 100, performance. Additional feature of the structure forming the waveguide are the coil cores 12, with its magnetic steel material of A848, but with permeability “A” composition. Special care is given to the geometry as well as the permeability of the parabolic shield antenna 18, where a dual function are defined in the design by first, the ability to collect the stray magnetic fields emanating from the EM generators 17.1-17.8, and secondly, the mechanical/structural supports it provides the waveguide 100 assembly. FIG. 17B further illustrates the incorporation of the coil-core 12, with its material permeability “C”, combined with the poleface 4.1 and/or 4.2 and is formed out of material composition “C”, while its ring insert 4.3x_{y}, forming the modified aperture 50, so as to bias the flux lines geometry in an anisotropic vector of magnitude and direction as a function of the AP to DP projected path, 400. This effect is due to the material composition and permeability of A848 composition “B”.

FIG. 17C is an isometric view of the waveguide segments and further elaboration of the waveguide construction, whereby the electromagnetic coils 17.1 and 17.5 are added to the core 12, the view further indicates the relative orientations of the polefaces 4.1 and 4.5, respectively. The orientations of the poleface 4.1 and 4.5 are in accordance with the rules 300, that govern the performance of electromagnetic radiation, under Maxwell formalism and as modified by the wave equation for forming a shaped field 400. The resulting effects of the waveguide 100, with its regulator 500, allow the apparatus to generate magnetic fields geometries on demand, while shifting the magnetic flux density axis based on the AP to DP travel path. The figures further indicate the relative locations of the parabolic antenna shield 18, the magnetic circuit return path structure 25, the poleface 4.1 and 4.5 as well as the ring insert 4.3x₁ which form the magnetic aperture 50.

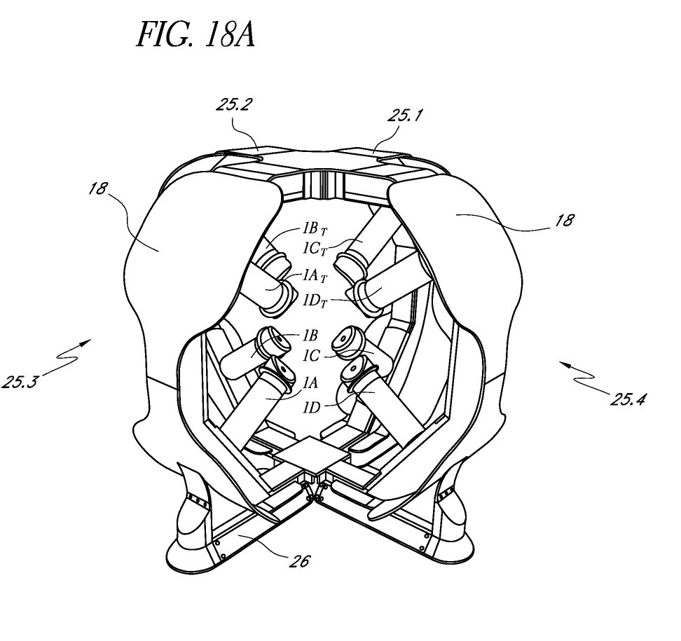

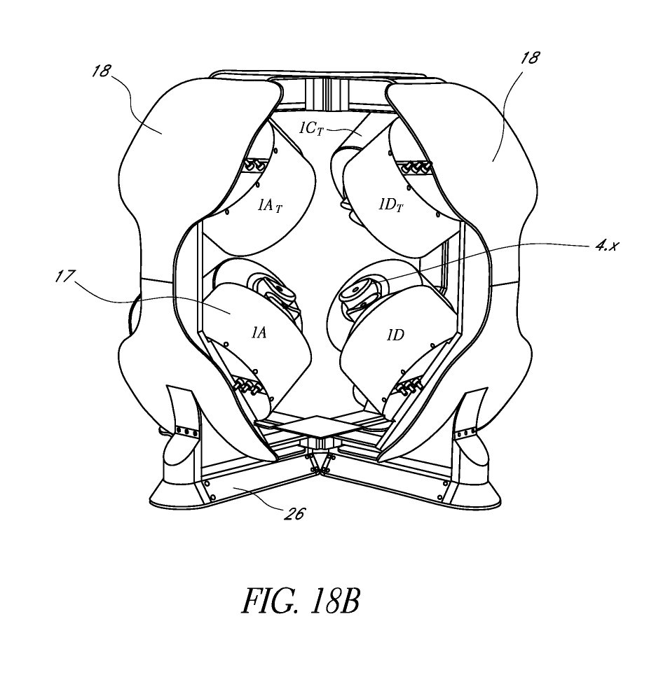

FIGS. 18A and 18B are isomorphic depictions of the waveguide assembly formed out of four segments 25.1, 25.2, 25.3, and 25.4, which are combined to form the spherical chamber 10 (effective space). Where the cores: 12.1, 12.2, 12.3, and 12.4, hold the coils: 1A, 1B, 1C and 1D in the scale model 1, and 17.1-17.8 in the waveguide 100, respectively. The four upper cores: 12.5, 12.6, 12.7 and 12.8 are the elements which hold the coils: 1A_{T} , 1B_{T}, 1C_{T} and 1D_{T} respectively. The structure geometry and the orientation of the cores relative to the chamber central axis is defined in accordance with the spherical topology which allows a linear solutions to the regulator 500. Computing the necessary PDE solutions by the regulator 500, and when establishing an optimal/ numerical commands 300, in order to guide and control the movements of the catheter distal end 7, from AP to DP. The spherical topology (see FIG. 13C) used in one or more embodiments provides for the formation of anisotropic EM wave propagation without the customary noni-liniear representation of the fields, which resulted in the inefficient and time consuming use of numerical as well a finite element (FEA) modeling of the field instead of the use of analytical modeling.

As described herein, the use of a substantially spherical arrangement of the cores 12.x linearizes aspects of the calculation of the currents in the magnet coils and thus simplifies the process of computing the currents needed to produce the desired field. This linearization also stabilizes operation of the device by reducing and/or avoiding nonlinearities that would otherwise make control of the desire field (and thus the catheter) difficult or impractical. The shaping of the magnetic field provided by the variations of permeability and the cores 12.x and provided by the shaping of the pole faces (e.g., the poleface 51) further improves the shape of the field and thereby reduces nonlinearities that would otherwise make such control difficult or impractical. Moreover, the shaping of the magnetic field provided by the variations of permeability and the cores 12.x and provided by the shaping of the pole faces (e.g., the poleface 51) further improves the shape of the field and increases the field strength of desired portions of the field in the region 10 and thus increases the efficiency and effectiveness of the system.

FIG. 18B is an isometric representation of the waveguide 100 where the entire assembly is shown and where the EM radiators 17.1-17.8, are placed. The entire structure is defined so as to integrate the topological as well as electrical functions whereby the mechanical integrity (stress and load characteristics associated with the size and weight of the EM radiators, as well as the magnetic forces which pull and push the structure are accented when designing such waveguide) and the magnetic circuits were optimized. The architecture of the magneto-optical wave guide, were the substantial elements of magnetic wave formation with optimal field density are combined to form an integrated and efficient guide for controlling medical device 7, movements within a patient body without the limitations noted by the prior art.

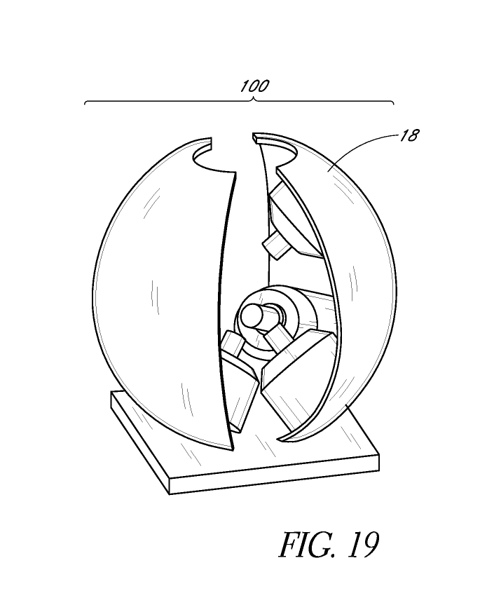

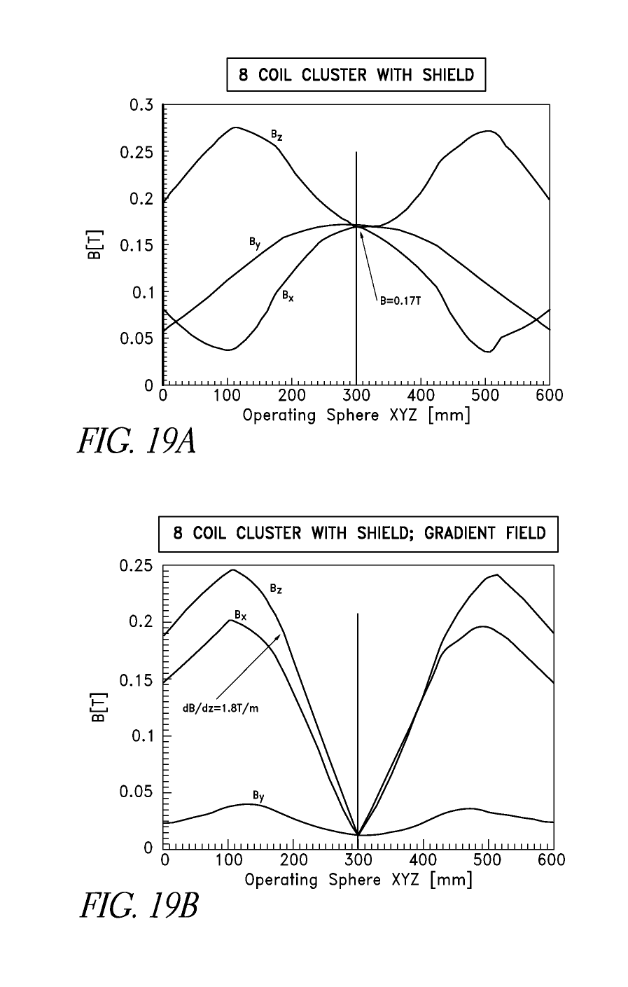

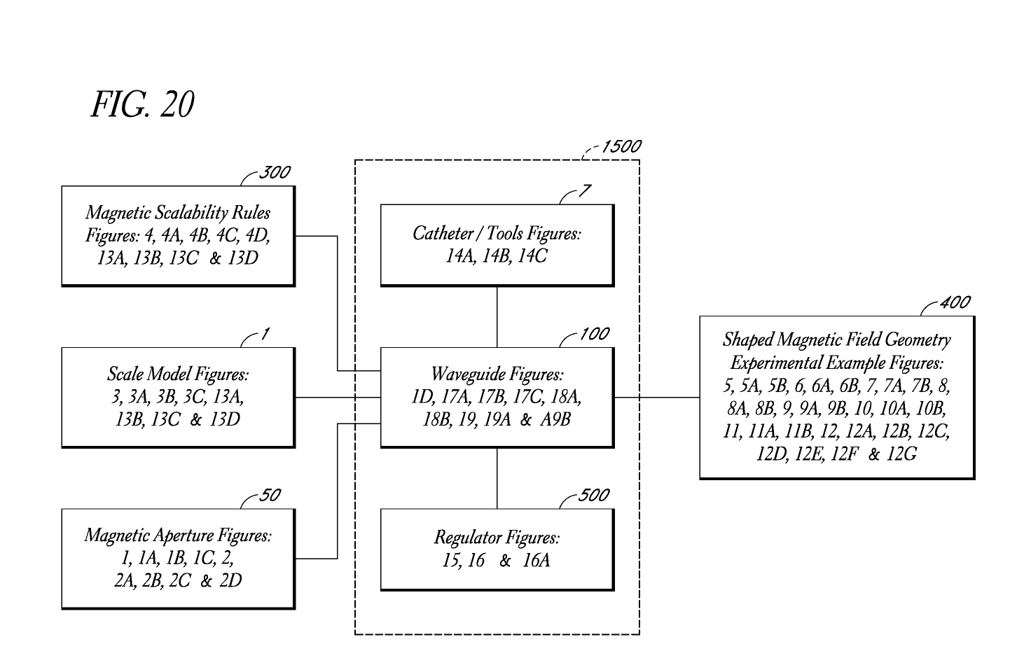

FIG. 19 illustrates the waveguide 100, and its 8 coils (coils 17.1-17.8) clustered and provided with an antenna shield 18. FIGS. 19, 19A and 19B illustrate the waveguide 100, configuration when the coil clusters 25.1-25.4 is fitted with the parabolic flux return shields 18. The eight-coil configuration and magnetic circuit is further enhanced by the use of such parabolic shields 18, to collect the stray magnetic flux radiated above and beyond the effective boundaries. In one embodiment the antenna shield 18 has a substantially spherical shape. In one embodiment the antenna shield 18 has a substantially parabolic shape. The antenna shield 18 is constructed of a ferromagnetic material to help contain the magnetic fields produced by the electromagnets and thus provide magnetic shielding to equipment and personnel outside the antenna shield 18. In one embodiment, the antenna shield 18 substantially encloses the volume occupied by the electromagnets (with appropriate breaks and gaps to allow for access to the region inside the shield 18).

FIGS. 19A and 19B are illustrations of the topological transformations as they alter the maximum field strength and field gradient. The transformation performed on scale model 1, from one iteration to the next, while assuming similar conditions as to power and coil size and evaluates the transformations relative to torque control field variations 303, in the magnetic center 10. The actions of the transformation further demonstrate the improvements associated with the use of parabolic antenna shield 18. As shown in FIG. 19A, in the eight coil cluster with shield 18, the magnetic B field 303, is symmetric and B=0.173 Tesla. FIG. 19B shows that in the eight coil cluster configuration with shield 18, produce a gradient field mode 304, which is symmetric and with a dB/dz=1.8 Tesla/m. The shielding produced by the parabolic antennas 18, is such that with a B field of 20 gauss to 2 Tesla, the effective perimeter magnetic field is less than 20 gauss 12″ away from the waveguide apparatus 100. The effective mass of the shield 18 further improves the overall magnetic circuit and improves the magnetic circuit.

FIG. 20 is a block diagram describing the relation between the functional elements of one or more embodiments. The scalability rules 300, guiding the behavior of the waveguide scale model 1, and the construction of the waveguide 100, are the results of identifying the boundary conditions to form a magnetic chamber efficiently: whereby the field magnitude and direction is further modified by the use of complex permeability technique and apparatus (the magnetic aperture geometry and the composition of material permeabilities). The technique of generating the shaped magnetic field is further improved by the use of ferro-magnification modality. The filed flux density efficiency is then improved by shifting the magnetic flux lines to form a magnetic density map, for the purpose of moving a permanent magnetic element 7, from its AP 5 state to its DP 6 state.

In forming the scalability rules, various electromagnetic effects were considered such as ferro-magnetic reflection, complex permeability of different materials as their effects on the geometry of the field are accounted. These efforts further lead to a description of a linear regulator 500, which performs the tasks of translating the necessary commands to form the magnetic map by using the rules and algorithm 300, to a set of EM generators 17.1-17.8, that shift the field flux-density-axis relative to the appropriate path for the permanent magnet 7, from AP to DP. The efforts of generating the appropriate magnetic field magnitude and direction is improved by the use of the magnetic aperture 50, which as noted above alter the field geometry of the shaped magnetic field 400.

FIG. 20 further delineates the relation between the waveguide and its rules of construction as well as the relation to the regulator 500, which act, interpret and executes the command structure to initiate the formations of specific field B and field gradient dB, simultaneously so as to move 303, rotate 304, and translate 305, the permanent magnetic element 7, from its AP 5, to its desired destination DP 6, with the heuristic regulatory command of optimal power setting when performing such tasks.

It is to be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but can be used alone or combined in other combinations. The excision of any disclosed element of the invention is explicitly contemplated as within the scope of the invention.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense, an equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a sub combination or variation of a sub combination.

Insubstantial changes from the claimed subject matter as viewed by a person with ordinary skill in the art, now known or later devised, are expressly contemplated as being equivalently within the scope of the claims. For example, although the specification above generally refers to a ferrous substance, one of ordinary skill in the art will recognize that the described ferrous substances can typically be any suitable magnetic material such as, for example a ferrous substances or compounds, nickel substances or compounds, cobalt substances or compounds, combinations thereof, etc. Therefore, obvious substitutions now or later known to one with ordinary skill in the art are defined to be within the scope of the defined elements. Accordingly, the invention is limited only by the claims.

What is claimed is:

1. An apparatus for controlling the movement of a catheter-type tool inside a body of a patient, comprising:
a magnetic field source for generating a magnetic field, said magnetic field source comprising a first coil disposed to produce a first magnetic field in a first magnetic pole piece and a second coil disposed to produce a second magnetic field in a second magnetic pole piece, said first magnetic pole piece comprising a first anisotropic permeability that shapes said first magnetic field, said second magnetic pole piece comprising a second anisotropic permeability that shapes said second magnetic field, said first magnetic pole piece and said second magnetic pole piece disposed to produce a shaped magnetic field in a region between said first magnetic pole piece and said second magnetic pole piece; and
a system controller for controlling said magnetic field source to control a movement of a distal end of a catheter, said distal end responsive to said magnetic field, said controller configured to control a current in said first coil, a current in said second coil, and a position of said first pole with respect to said second pole.

2. The apparatus of claim 1, said system controller comprises a closed-loop feedback servo system.

3. The apparatus of claim 1, said first magnetic pole piece comprising a body member and a field shaping member, said field shaping member disposed proximate to a face of said first pole piece, said body member comprising a first magnetic material, said field shaping member comprising a second magnetic material different from said first magnetic material.

4. The apparatus of claim 1, said first magnetic pole piece comprising a body member and a field shaping member, said field shaping member disposed proximate to a face of said first pole piece, said body member comprising a first magnetic material composition, said field shaping member comprising a second magnetic material different from said first magnetic material composition.

5. The apparatus of claim 4, wherein said second magnetic material composition comprises an anisotropic permeability.

6. The apparatus of claim 1, wherein said first magnetic pole piece comprises a face comprising a concave depression.

7. The apparatus of claim 1, wherein said first magnetic pole piece comprises a face having a first concave depression and said second magnetic pole piece comprises a face having a second concave depression, said shaped field formed in a region between said first concave depression and said second concave depression.

8. The apparatus of claim 1, wherein said first magnetic pole piece comprises a core member comprising a first magnetic material composition and a poleface member disposed about said magnetic core comprising a second magnetic material composition.

9. The apparatus of claim 8, wherein said poleface member is substantially cylindrical.

10. The apparatus of claim 1, wherein said first magnetic pole piece comprises a substantially cylindrical core comprising a first magnetic material composition and a poleface cylinder disposed about said magnetic core comprising a second magnetic material composition.

11. The apparatus of claim 1, wherein said substantially cylindrical core extends substantially a length of said first magnetic pole piece.

12. The apparatus of claim 11, wherein a cylindrical axis of said first magnetic pole piece is disposed substantially parallel to a cylindrical axis of said second magnetic pole piece.

13. The apparatus of claim 1, wherein said distal end comprises a permanent magnet.

14. The apparatus of claim 1, wherein said distal end comprises an electromagnet.

15. The apparatus of claim 1, wherein said distal end comprises a first magnet having a first coercivity and a second magnet having a second coercivity.

16. The apparatus of claim 1, wherein said first magnetic pole piece comprises a first magnetic material and wherein said system controller comprises a control module to control a permeability of said first magnetic material.

17. The apparatus of claim 1, further comprising an operator interface unit.

18. The apparatus of claim 1, wherein said servo system comprises a correction factor that compensates for a dynamic position of an organ, thereby offsetting a response of said distal end to said magnetic field such that said distal end moves in substantial unison with said organ.

19. The apparatus of claim 18, wherein said correction factor is generated from an auxiliary device that provides correction data concerning said dynamic position of said organ, and wherein when said correction data are combined with measurement data derived from said sensory apparatus to offset a response of said servo system so that said distal end moves substantially in unison with said organ.

20. The apparatus of claim 19, wherein said auxiliary device is at least one of an X-ray device, an ultrasound device, and a radar device.

21. The apparatus of claim 1, wherein said system controller includes a Virtual Tip control device to allow user control inputs.

22. The apparatus of claim 1, further comprising:
first controller to control said first coil; and
a second controller to control said second coil.

23. The apparatus of claim 11, wherein said first controller receives feedback from a magnetic field sensor.

24. The apparatus of claim 1, wherein said system controller coordinates flow of current through said first and second coils according to inputs from a Virtual Tip.

25. The apparatus of claim 14, wherein said Virtual Tip provides tactile feedback to an operator when a position error exceeds a threshold value.

26. The apparatus of claim 24, wherein said Virtual Tip provides tactile feedback to an operator according to a position error between an actual position of said distal end and a desired position of said distal end.

27. The apparatus of claim 24, wherein said system controller causes said distal end to follow movements of said Virtual Tip.

28. The apparatus of claim 24, further comprising:
a mode switch to allow a user to select a force mode and a torque mode.

29. An apparatus for controlling the movement of a catheter-like tool to be inserted into the body of a patient, comprising:
a controllable magnetic field source having a first cluster of poles and a second cluster of poles, wherein at least one pole in said first cluster of poles comprises an anisotropic pole piece, said anisotropic pole piece comprising a core member and a poleface member, said core member and said poleface member comprising different compositions of magnetic material, said first cluster of poles and said second cluster of poles disposed to direct a shaped magnetic field in a region between said first cluster of poles and said second cluster of poles;
a first group of electromagnet coils provided to said first cluster of poles and a second group of electromagnet coils provided to said second cluster of poles; and
a controller to control electric currents in said first group of electromagnet coils and said second group of electromagnet coils to produce said shaped magnetic field.

30. The apparatus of claim 29, wherein said poleface member comprises a substantially concave face.

31. The apparatus of claim 29, wherein said controller controls a permeability of said poleface member.

32. The apparatus of claim 29, further comprising an operator interface unit.

33. The apparatus of claim 29, wherein said first cluster of poles is coupled to said second cluster of poles by a magnetic material.

34. A method for controlling movement of a tool having a distal end to be inserted in a body, comprising:
calculating a desired direction of movement for said distal end;
computing a magnetic field needed to produce said movement, said magnetic field computed according to a first bending mode of said distal end and a second bending mode of said distal end;
controlling a plurality of electric currents and pole positions to produce said magnetic field; and
measuring a location of said distal end.

35. The method of claim 34, further comprising controlling one or more electromagnets to produce said magnetic field.

36. The method of claim 34, further comprising simulating a magnetic field before creating said magnetic field.

37. An apparatus for controlling the movement of a catheter-like tool having a distal end responsive to a magnetic field and configured to be inserted into the body of patient, comprising:
a magnetic field source for generating a magnetic field, said magnetic source comprising an electromagnet, said electromagnet comprising:
an electromagnet coil;
a pole piece core; and
a poleface insert, said poleface insert having a different permeability than said pole piece core;
a sensor system to measure a location of said distal end;
a sensor system to measure positions of a plurality of fiduciary markers;
a user input device for inputting commands to move said distal end; and
a system controller for controlling said magnetic field source in response to inputs from said user input device, said radar system, and said magnetic sensors.

38. The apparatus of claim 37, said system controller comprising a closed-loop feedback servo system.

39. The apparatus of claim 37, wherein said poleface insert is disposed proximate to a face of said pole piece core.

40. The apparatus of claim 37, said distal end comprising one or more magnets.

41. The apparatus of claim 37, said distal end comprising a first magnet having a first coercivity and a second magnet having a second coercivity.

42. The apparatus of claim 37, wherein said system controller calculates a position error and controls said magnetic field source to move said distal end in a direction to reduce said position error.

43. The apparatus of claim 37, wherein said system controller computes a position of said distal end with respect to a set of fiduciary markers.

44. The apparatus of claim 37, wherein said system controller synchronizes a location of said distal end with a fluoroscopic image.

45. The apparatus of claim 37, further comprising an operator interface unit.

46. The apparatus of claim 37, wherein a correction input is generated by an auxiliary device that provides correction data concerning a dynamic position of an organ, and wherein said correction data are combined with measurement data from said radar system to offset a response of said control system so that said distal end moves substantially in unison with said organ.

47. The apparatus of claim 46, wherein said auxiliary device comprises at least one of an X-ray device, an ultrasound device, and a radar device.

48. The apparatus of claim 46, wherein said user input device comprises a virtual tip control device to allow user control inputs.

49. The apparatus of claim 37, further comprising a virtual tip with force feedback.

50. The apparatus of claim 37 wherein a first coil cluster is fitted with shield for flux return.

51. The apparatus of claim 37, further comprising a boundary condition controller, and wherein computing the fields in the surroundings of the catheter based on the fields on 2D planes.

52. The apparatus of claim 37, further comprising a user interface control to switch from torque control to force control.

53. The apparatus of claim 37, wherein said system controller is configured to produce coil current polarities and magnitudes are generated to produce desired field directions for torque and force field is established.

54. The apparatus of claim 37, a low level logic simulation of action is provided.

55. An apparatus for controlling the movement of a catheter-type tool inside a body of a patient, comprising:
a magnetic field source for generating a magnetic field, said magnetic field source comprising a first coil disposed to produce a first magnetic field in a first magnetic pole piece and a second coil disposed to produce a second magnetic field in a second magnetic pole piece, said first magnetic pole piece comprising a first anisotropic permeability that shapes said first magnetic field wherein a permeability of a first portion of said first magnetic pole piece is less than a permeability of a second portion of said first magnetic pole piece, wherein said second portion is relatively closer to a centerline of said first pole piece than said second portion, said first magnetic pole piece and said second magnetic pole piece disposed to produce a shaped magnetic field in a region between said first magnetic pole piece and said second magnetic pole piece; and
a system controller for controlling said magnetic field source to control a movement of a distal end of a catheter, said distal end responsive to said magnetic field, said controller configured to control a current in said first coil, a current in said second coil, and a position of said first pole with respect to said second pole.

56. The apparatus of claim 55, further comprising a magnetic shield disposed about said magnetic field source.

57. The apparatus of claim 55 further comprising a magnetic shield disposed about said magnetic field source, said magnetic shield substantially enclosing a volume occupied by said magnetic field source.

58. The apparatus of claim 55, wherein said anisotropic permeability is produced by changes in chemical composition of different regions of said first magnetic pole piece.

59. The apparatus of claim 55, wherein said anisotropic permeability is produced by constructing said pole piece from a plurality of members having different magnetic permeability.

60. The apparatus of claim 55, wherein said anisotropic permeability is produced by constructing said pole piece from three or more members having different magnetic permeability.

61. The apparatus of claim 55, further comprising at least eight magnetic pole pieces disposed in a substantially spherical arrangement about a sphere to produce a magnetic field region proximate to a center of said sphere.
